(19) United States
(12) Patent Application Publication
(10) Pub. No.: US 2024/0417677 A1
(43) Pub. Date: Dec. 19, 2024

(54) CANOLA MEAL EXTRACTS FOR MICROBIAL FERMENTATION

(71) Applicant: University of Saskatchewan, Saskatoon (CA)

(72) Inventors: Bishnu Acharya, Saskatoon (CA); Nirpesh Dhakal, Saskatoon (CA)

(21) Appl. No.: 18/745,228

(22) Filed: Jun. 17, 2024

Related U.S. Application Data
(60) Provisional application No. 63/521,236, filed on Jun. 15, 2023.

(51) Int. Cl. C12N 5/00; C12N 1/16; C12N 1/20; C12N 9/64; C12N 9/76

(52) U.S. Cl. CPC C12N 5/0018; C12N 1/16; C12N 1/20; C12N 9/6427; C12N 9/6481; C12N 2500/14; C12N 2500/16; C12N 2500/22; C12N 2500/24; C12N 2500/34; C12N 2500/36; C12N 2500/38; C12N 2500/74; C12N 2500/76; C12N 2500/80; C12Y 304/21004; C12Y 304/21062; C12Y 304/22002; C12Y 304/23001

(57) ABSTRACT

The disclosure relates to canola meal extracts (CMEs), and methods of making said CMEs. Also provided herein are compositions and culture media comprising said CMEs and use of said CMEs, compositions, and culture media for microbial fermentation across a broad class of microbes. The disclosure further relates to use of said CMEs and compositions as a partial or complete replacement for other organic extracts such as yeast extract.

### FIELD

The disclosure relates to canola meal extracts for use as culture medium supplements for microbial culture or microbial fermentation, and methods of making and kits comprising the same.

### BACKGROUND

Microbial bioprocess often requires undefined media supplements to enhance the growth and productivity of several microbial species. These are termed ‘undefined’ as their nutritional composition may be poorly characterized, even while their use as a supplement in media fulfills the demand for one or more nutrition elements in the form of an organic source of nitrogen, vitamins, minerals, and trace metals (Arora, 2022; Emerson and Tang, 2007; Sperotto et al., 2021). Conventionally, yeast extract (YE) supplement often provides the prerequisites for microbial nutrition during microbial fermentation (Qiang, 2019; Sperotto et al., 2021). YE is considered among the more costly ingredients used in microbial media, with market prices ranging from $100 to $300 per kg, adding to the overall cost of fermentation.

Currently, canola meal (CM) is positioned as substandard biomass based on its very low market value, which is as low as $450 per metric ton (Canola Council, 2022), although being nutritionally rich. Although the total content of nutrients varies between YE and CM, they have similar macro and micro-nutrient compositions in terms of amino acids, vitamins, and minerals (Vieira et al., 2016; Wickramasuriya et al., 2015). CM contains protein up to 42% of its dry mass with a standard profile for the amino acids, which acts as a good source of organic nitrogen (Canola Council, 2019). Although lysine is limited compared to YE, CM has a substantial amount of methionine. The reported protein efficiency ratio (PER) of CM is 4.2, which is greater than that of YE (PER=2.4) (Boling-Frankenbach et al., 2001; Vieira et al., 2016). Compared to YE, vitamin ranges in CM are excellent, with concentrations of B3, B5, B6, B2, B9, B7, E, and choline at 160, 9.5, 7.2, 5.8, 2.3, 1.1, 20.9, and 6700 mg/kg of dry mass, respectively (Vieira et al., 2016; Wickramasuriya et al., 2015). This is especially notable, given that vitamins B5, B7, B2, E, and choline are limited in YE. Along with the amino acids, vitamins (mostly vitamin B ranges) are rate-limiting nutrients for microbial growth and productivity, as they act as coenzymes for metabolic enzymes (Emerson and Tang, 2007; Sperotto et al., 2021), indicating that an abundance of these nutritional elements sustains elevated microbial growth rates. CM also contains minerals and trace metals in the form of calcium, phosphorus, sodium, chlorine, potassium, sulfur, magnesium, copper, iron, manganese, molybdenum, zinc, selenium, etc., and their content is comparable with YE (Vieira et al., 2016; Wickramasuriya et al., 2015). Moreover, it is a good source of carbohydrates, containing 4-6% of cellulose and 13-16% of non-cellulosic polysaccharides. Fermentable sugars could be hydrolyzed from these carbohydrates as an essential energy source for microbial media.

Although the majority of the above nutrients are water extractable, there has been little focus on how to optimize nutrient extraction from CM using this extraction method. Developing and optimizing canola meal extract (CME) suitable for microbial media supplementation from the substandard CM biomass could result in increased utilization and valorization of this abundant biomass.

Rapeseed, a close cultivar to canola, has been investigated for extract development from its meal by the Thunen-Institute of Agricultural Technology, Germany (Kuenz, 2021). It was reported that this extract can replace about 80%-100% of yeast extract for L-lactic acid and 1,3-propanediol production, respectively. However, unlike the abundance of vitamin B in CM, vitamin B ranges and amino acids, such as cysteine and tryptophan, are limited in the so-developed rapeseed meal extract and need to be supplemented separately.

There has been substantially less effort to develop CM into microbial media, however work comparing canola protein with soybean protein demonstrated that CM has a better amino acid balance than does soybean protein (Maenz, 2007). The soy protein digest, soytone, has established commercial application in microbial bioprocessing, indicating that optimized CME may perform as a low-cost substitute, or an improvement upon, soytone.

### SUMMARY

Canola meal (CM) is a substandard yet abundant and inexpensive biomass that is produced as a byproduct of the canola processing industry. Recently, there has been growing interest in developing improved commercial uses for CM. Using this substandard and inexpensive biomass, the present inventors derived a canola meal extract that has a high-value nutritional profile that is suitable for a wide variety of applications.

Accordingly, provided herein is a canola meal extract (CME) comprising 20-80% protein, 10-30% carbohydrates, 5-30% fermentable sugars, 1-10% lipids, 1-10% minerals, and 50-300 μg phenolics per g dry weight.

In one embodiment, the CME further comprises 50-200 mg of vitamins per kg dry weight.

In another embodiment, the minerals comprise one or more selected from the group consisting of P, K, S, Se, Cu, Fe, Mn, Ca, Mg, and Zn.

In another embodiment, the vitamins comprise one or more selected from the group consisting of vitamin K, niacin, riboflavin, thiamine, biotin, pyridoxine, panthothenate and choline.

In another embodiment, the CME further comprises less than 2% other impurities.

Also provided herein is a culture medium comprising the CME as described herein.

In one embodiment, the culture medium comprises one or more additional extracts, optionally wherein the one or more additional extracts comprise yeast extract, beef extract, peptone, tryptone, or soytone, or a combination thereof.

Also provided herein is a use of the CME or the culture medium as described herein in a culture with at least one microbe, for promoting microbial growth or survival.

Further provided is a method of promoting microbial growth or survival comprising contacting at least one microbe with the CME or the culture medium as described herein.

Also provided herein is a use of the CME or the culture medium as described herein in a culture with at least one mammalian cell, for promoting growth or survival of the at least one mammalian cell.

Further provided is a method of promoting growth or survival of at least one mammalian cell comprising contacting the at least one mammalian cell with the CME or the culture medium as described herein.

In one embodiment, the at least one microbe comprises a bacterium, an archaeum, a fungus, an alga, or a protozoan, or a combination thereof.

In another embodiment, the bacterium comprises a carboxydotroph, optionally of genus Cupriavidus, optionally Cupriavidus necator,
the bacterium comprises an acetogen, optionally of genus Clostridium, optionally Clostridium carboxidivorans;
the bacterium produces lactic acid, optionally wherein the bacterium comprises the genus Lactobacillus, Leuconostoc, Pediococcus, Lactococcus, or Streptococcus or combinations thereof, optionally Lactobacillus rhamnosus or Lactobacillus acidophilus;
the bacterium comprises a coliform, optionally of genus Escherichia, optionally Escherichia coli;
the fungus comprises an oleaginous fungus, optionally of genus Mortierella, optionally Mortierella alpina fungus comprises genus Saccharomyces, optionally Saccharomyces cerevisiae; or
the fungus comprises a saccharifying mold, optionally of genus Aspergillus, optionally Aspergillus oryzae, or Aspergillus niger, or a combination thereof.

Also provided herein is a method of producing a canola meal extract (CME) comprising:
combining canola meal with a fluid to form a mixture;
optionally raising the pH of the mixture to between 9 and 12;
heating the mixture to a temperature between 20° C. and 170° C.;
catalyzing the mixture; and
obtaining the CME from the mixture.

In one embodiment, raising the pH of the mixture comprises adding a base, optionally NaOH, to the mixture.

In another embodiment, catalyzing comprises treating the mixture with at least one enzyme, wherein the at least one enzyme comprises a proteolytic enzyme.

In another embodiment, the proteolytic enzyme comprises papain, pepsin, alkaline protease, optionally Alcalase, trypsin, or a combination thereof.

In another embodiment, catalyzing comprises treating the mixture with the at least one enzyme for between 2 and 96 hours, optionally with continuous agitation, optionally at a temperature between 20° C. and 90° C. and optionally between pH 3 and 12.

In another embodiment, catalyzing comprises treating the CME with at least one fungus, optionally of genus Aspergillus, optionally Aspergillus oryzae, Mortierella, or a combination thereof, optionally for between 2 and 7 days.

In another embodiment, the catalyzing comprises inherent lactic fermentation.

In another embodiment, the step of catalyzing is performed prior to the steps of optionally raising the pH of the mixture and heating the mixture.

In another embodiment, the steps of optionally raising the pH of the mixture to between 9 and 12 and heating the mixture to a temperature between 20° C. and 170° C. are performed under continuous agitation.

In another embodiment, the method comprises at least one pretreatment step, optionally defatting or sterilization of the canola meal.

Further provided is the produced by a method as described herein.

Also provided is a kit comprising a CME or a composition as described herein and a culture medium.

In one embodiment, the kit further comprises one or more extracts, optionally wherein the one or more extracts comprise yeast extract, beef extract, peptone, tryptone, soytone, or a combination thereof.

Also provided herein is a canola meal extract (CME) comprising 20-65% protein, 5-30% fermentable sugars, 1-10% lipids, 5-8% minerals, and 50-200 mg of vitamins per kg dry weight.

In an embodiment, the CME further comprises less than 300 μg phenolics per g dry weight.

In an embodiment, the phenolics comprise sinapic acid.

In an embodiment, the CME further comprises less than 5% water-soluble hemicellulose per dry weight.

In an embodiment, the CME further comprises less than 2% other impurities.

In an embodiment, the culture medium further comprises micronutrients, optionally vitamins, essential minerals, or trace metals; macronutrients, optionally essential amino acids; or combinations thereof.

In an embodiment, a CME, a composition, or a culture medium herein disclosed promotes microbial growth or survival.

In an embodiment is a use of a CME, a composition, or a culture medium herein disclosed in a culture with at least one microbe, for promoting microbial growth or survival.

In an embodiment, a CME, a composition, or a culture medium herein disclosed promotes growth or survival of a mammalian cell culture.

In an embodiment is a use of a CME, a composition, or a culture medium herein disclosed in a culture with at least one mammalian cell, for promoting growth or survival of the at least one mammalian cell.

In an embodiment is a use herein disclosed for microbial fermentation.

In an embodiment, the culture produces polymer, optionally polyhydroxyalkanoates; lipids, optionally polyunsaturated fatty acids, optionally arachidonic acid; proteins, optionally enzymes or recombinant proteins; organic acids, optionally lactic acid; alcohol, optionally ethanol; bacterial carbohydrates, optionally cellulose; fuels, optionally hydrogen; antioxidants; polyphenols; or flavonoids; or combinations thereof.

Also provided herein is a method of producing a canola meal extract (CME) comprising:
i) combining finely milled or ground canola meal with a fluid to form a mixture, wherein the mixture has a pH between 5 and 12;
ii) heating the mixture to a temperature between 25° C. and 160° C.; and
iii) extracting the CME from the heated mixture.

In an embodiment, the extracting comprises water extraction or alkaline extraction.

In an embodiment, the extracting comprises alkaline extraction.

In an embodiment, the extracting is performed at a pH between 9.0 and 12.0.

In an embodiment, the extracting is performed at a temperature between 60° C. and 160° C.

In an embodiment, the extracting comprises continuous agitation for 1 to 5 hours.

In an embodiment, a method described herein further comprises a step of re-extraction.

In an embodiment, a method described herein further comprises at least one pretreatment step, at least one post-treatment modification step, or a combination thereof.

In an embodiment, the at least one pretreatment step comprises treating the canola meal with hexane, acidic ethanol, Aspergillus-based pre-digestion or a combination thereof.

In an embodiment, the at least one post-treatment modification step comprises hydrolysis of macromolecules, optionally carbohydrates or proteins.

In an embodiment, the hydrolysis comprises an acidic treatment, an enzymatic treatment, a microbial treatment, or a combination thereof.

In an embodiment, the acidic treatment comprises treating the CME with hydrochloric acid, optionally at a pH between 1 and 4, optionally at a temperature between 60° C. and 121° C., optionally for 0.5 to 2 hours.

In an embodiment, the enzymatic treatment comprises treating the CME with at least one enzyme, wherein the at least one enzyme comprises a proteolytic enzyme, a cellulolytic enzyme, or a combination thereof.

In an embodiment, the proteolytic enzyme comprises papain, pepsin, or alkaline protease, or a combination thereof.

In an embodiment, the cellulolytic enzyme comprises cellulase, hemicellulase, or a combination thereof.

In an embodiment, the enzymatic treatment comprises treating the CME with the at least one enzyme for between 24 and 96 hours, optionally with continuous agitation, optionally at a temperature between 25° C. and 90° C.

In an embodiment, the microbial treatment comprises treating the CME with at least one fungus, optionally of genus Aspergillus, Mortierella, or a combination thereof, optionally for between 2 and 7 days.

In an embodiment, is a CME, or a composition herein disclosed, for use as a partial replacement or complete replacement for a nitrogen or organic extract, optionally yeast extract, beef extract, peptone, tryptone, soytone or a combination thereof.

In an embodiment, the CME or composition herein disclosed is freeze dried or spray dried.

In an embodiment, the CME, the composition, or the culture medium herein disclosed is food grade.

Also provided herein is a kit comprising a CME or a composition herein disclosed, culture medium, and a leaflet containing instructions to produce a supplemented culture medium.

In an embodiment, the CME or the composition in the kit is freeze dried or spray dried.

In an embodiment, the kit further comprises one or more organic extracts, optionally wherein the one or more organic extracts comprise yeast extract, beef extract, peptone, tryptone, soytone, or a combination thereof.

In an embodiment, the kit further comprises micronutrients, optionally vitamins, essential minerals, or trace metals; macronutrients, optionally essential amino acids; or combinations thereof.

These and other features and advantages of the present disclosure will become apparent from the following detailed description taken together with the accompanying drawings. It should be understood, however, that the detailed description and specific examples, while indicating preferred implementations of the present disclosure, are given by way of illustration only, since various changes and modifications within the spirit and scope of the disclosure will become apparent to those of skill in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows an estimation of total extracts (T.E.; left-most bar per triplet of bars), total protein (T.P; middle bar per triplet of bars), and total reducing sugar (T.R.S.; right-most bar per triplet of bars) from untreated (UT), defatted (DF), and acidic ethanol and defatted (AEOH-DF) canola meal (CM), produced using either water extraction (WE) or alkaline extraction (AE) at two different temperatures (25° C. and 75° C.) in an exemplary embodiment of the disclosure.

FIG. 2 shows steps of media preparation in an exemplary embodiment of the disclosure. A) is canola meal (CM); b) is liquid canola meal extract (CME) prepared from a); c) is freeze dried CME prepared from b); d) is CME in media prepared from c); and e) & f) are shake flask studies for CME supplemented media with 50% I & 100% (f) yeast extract (YE) replacement, including comparison of water extracted CME, alkaline extracted CME, acidic ethanol treated alkaline extracted CME and YE and minimal salt media (MSM).

FIG. 3 shows the effect of partial CME substitution on the growth of gram negative aerobic bacteria at 75° C. in an exemplary embodiment of the disclosure. Growth profile (top), specific growth rate (bottom left), and percentage polyhydroxyalkanoates (PHA) production (bottom right) of Cupriavidus necator cultured in DSMZ 21 media with glucose are shown; DSMZ 21 is supplemented with: 1 g of yeast extract (YE); 0.5 g YE and 0.5 g water extracted CME (WE:YE (1:1)); 0.5 g YE and 0.5 g alkaline extracted CME (AE:YE (1:1)); and 0.5 g YE and 0.5 g acidic ethanol treated alkaline extracted CME (AEOH:YE (1:1)). Statistics were Tukey's Honestly Significant Difference (HSD) test at 95% confidence intervals, wherein “a” is significantly different than “b”.

FIG. 4 shows the effect of total CME substitution on the growth of gram negative aerobic bacteria at 75° C. in an exemplary embodiment of the disclosure. Growth profile (top), specific growth rate (bottom left), and percentage polyhydroxy alkanoates (PHA) production (bottom right) of Cupriavidus necator cultured in DSMZ 21 media with glucose are shown; DSMZ 21 supplemented with: glucose (DSMZ 21), 1 g of YE (YE); 1 g equivalent of water extracted CME extract; and 1 g equivalent of alkaline extracted CME (AE). Statistics were Tukey's HSD test at 95% confidence intervals, wherein “a” is significantly different than “b” and both are significantly different than “c”.

FIG. 5 shows biomass accumulation (left; by mg dry mass) and lipid production (right; by % mean lipid in shake flask) in Mortierella alpina (M. aplina) when supplemented with CME and YE in shake flask cultures at 75° C. and a working volume of 50 ml in an exemplary embodiment of the disclosure. Normal media (N.M.) represents normal media with 100% YE supplement, sub-critically extracted (S.E.) represents 50% YE supplement and 50% replaced by CME supplement extracted sub-critically at 160° C., and alkaline extracted (A.E.) represents 50% YE supplement and 50% replaced by CME supplement extracted in alkaline pH.

FIG. 6 shows biomass accumulation (left; by mg dry mass) and lipid production (right; by % mean lipid in fermenter) in M. alpina when supplemented with CME and YE in a fermenter at a working volume of 1 L or 2 L at 75° C. in an exemplary embodiment of the disclosure. Normal media (N.M) represents normal media with 100% YE supplement, and alkaline extracted (A.E.) represents 50% YE supplement and 50% replaced by CME supplement extracted in alkaline pH.

FIG. 7 shows changes in the turbidity of the medium during growth of M. alpina in medium with alkaline (pH 10.5) CME at 50% and YE at 50% (top row) and 100% alkaline (pH 10.5) CME with no YE (bottom row) at 75° C. in an exemplary embodiment of the disclosure. Panels from left to right show day 1 culture, day 4 culture, and day 7 culture.

FIG. 8 shows a molecular weight analysis of CME through SDS PAGE. L-protein ladder (10 kDa to 250 kDa) WE_C-extraction with water with uncontrolled pH, WE_P-extraction with papain at pH 5.8, AE_C-alkaline extraction at pH 11, AE_P-alkaline extraction followed by papain treatment, AE_PC*-alkaline treated extract, AE_P3 & AE_P6-alkaline treated extract catalyzed by 5 mg and 10 mg of papain respectively, AO_WE & AO_AE-cultured in A. oryzae followed water and alkaline extraction respectively, SC_WE and SC_AE-subcritically extracted CM at 160° C. without pH treatment and at pH 11 respectively, Liq. and sp. 48 & 72-liquid and spray dried enzyme catalyzed CME after 48 and 72 hours of extraction. All enzyme extractions were done in 10 mM phosphate buffer pH 5.8, temperature 55° C., 250 rpm, and with an enzyme to biomass ratio of 1:250.

FIG. 9 shows a comparison of C. necator growth and PHA yield at 100% YE replacement. FIG. 9A shows a growth profile; FIG. 9B shows specific growth rates;

FIG. 9 C shows the percentage polyhydroxy alkanoates (PHA) yield. C. necator was cultured in Minimal salt medium (MSM); MSM supplemented with—2 g of YE (YE); 2 g YE protein equivalent of WE (WE); 2 g YE protein equivalent of AE (AE).

FIG. 10 shows a comparison of C. necator growth and PHA yield at 50% YE replacement. FIG. 10A shows a growth profile; FIG. 10B shows specific growth rates; FIG. 10 C shows the percentage polyhydroxy alkanoates (PHA) yield. C. necator cultured in MSM supplemented with: 2 g of YE (YE); 1 g YE and 1 g YE protein equivalent of WE (WE:YE (1:1)); 1 g YE and 1 g YE protein equivalent of df-AcEtOH (AEOH:YE (1:1)).

FIG. 11 shows growth curves of A) L. rhamnosus and B) L. acidophilus, and C) specific growth of lactic acid bacteria (LAB) cultured in standard MRS medium (Std.), MRS medium where YE was replaced equivalent weight of papain extracted CME (CanX+ME) and MRS medium where both YE and ME were replaced by equivalent weight of papain extracted CME (CanX).

FIG. 12 shows growth comparisons of Saccharomyces cerevisiae in different combinations of CME and commercial dry malt extract DME, pH 6 and temperature 32° C. Note: no extra sugar was supplemented during this trial.

FIG. 13 shows growth comparisons of Clostridium carboxidivoran when yeast extract in 100 ml standard PETC 1754 media (std) was replaced by CME at pH 5.9, 37° C., and 150 rpm.

FIG. 14 shows comparisons in biomass and lipid accumulation of M. alpina at 9th day of culture when YE was 50% replaced with sub-critically extracted (SE) and alkaline extracted (AE) CM grown in shake flask (100 rpm) at 28° C. Std: standard media with YE; SE:YE (1:1): standard media where YE was 50% replaced with SE; AE:YE (1:1): standard media where YE was 50% replaced with AE.

FIGS. 15A and B show comparisons in biomass and lipid accumulation of M. alpina at 9th day of culture when YE was a. 50% and b. 100% replaced with sub-critically extracted (SE) and alkaline extracted (AE) CM grown in 1-L and 2-L fermenters with 100 rpm at 28° C. and 2 vvm. Std.: standard media with YE; SE:YE (1:1): standard media where YE was 50% replaced with SE; AE:YE (1:1): standard media where YE was 50% replaced with AE; SE: standard media where YE was 100% replaced with SE; AE: standard media where YE was 100% replaced with AE.

FIGS. 16A and B show comparisons in biomass and lipid accumulation of the M. alpina at 9th day of culture when YE and glucose were 100% replaced with sub-critically extracted (SE-G) and alkaline extracted (AE-G) CM, whereas Std-G represents glucose omitted standard media. FIG. 16A shows a shake flask culture at 100 rpm and 28° C. and FIG. 16B shows a culture grown in 1 L fermenter with 100 rpm at 28° C. and 2 vvm.

FIG. 17 shows changes in medium appearance during growth of M. alpina in medium with AE:YE (1:1) (above) and AE (1:0) (below) replacement of YE. Left to right day 1 culture, day 4 culture, and day 7 culture.

FIG. 18A shows extraction of canola meal with and without enzymatic catalysis. FIG. 18B shows the solubility of 40 mg of freeze dried WE in 1 ml of water, where the turbidity or haze formation in the solution can be seen. FIG. 18C shows that bio-catalyzed extracts are less hazy when mixed with same amount in water. WE, AE, WE_P, AE_P, A.o E, and YE are water extract, alkaline extract, papain catalyzed in water, papain catalyzed after alkaline extraction, alkaline extraction after A. oryzae catalysis, and yeast extract, respectively.

FIG. 19 shows enzyme catalyzed canola meal has can have better properties as compared to non-catalyzed water or alkaline extracted canola meal. This can be seen in precipitation or increased turbidity in AE and WE, respectively, after sterilization in 121° C. for 15 min, whereas enzyme catalyzed extract remains clear without precipitation.

Further aspects and features of the example embodiments described herein will appear from the following description taken together with the accompanying drawings.

### DETAILED DESCRIPTION OF THE DISCLOSURE

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

### I. Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “Including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, 5-10%, or about 5% of the number to which reference is being made.

As used in this specification and the appended claims, the singular forms “a”, “an” and “the” include plural references unless the content clearly dictates otherwise. Only terms clearly indicated to the contrary, such as “only one of” or “exactly one of” or, when used in the claims, “consisting of” or “is” will refer to the inclusion of exactly one element. Thus, for example, a composition containing “a compound” includes a mixture of two or more compounds, and a culture containing a bacterium includes a mixture of two or more species of bacteria.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Although any methods and materials similar or equivalent to those described herein can also be used in the practice or testing of the present disclosure, examples of methods and materials are now described.

### II. Canola Meal Extracts

Canola meal (CM) is a substandard yet abundant and inexpensive biomass that is produced as a byproduct of the canola processing industry. Recently, there has been growing interest in developing improved commercial uses for CM. Using this substandard and inexpensive biomass, the present inventors derived a canola meal extract that has a high-value nutritional profile that is suitable for a wide variety of applications.

Accordingly, one aspect of the present disclosure is a canola meal extract (CME) comprising protein, carbohydrates, fermentable sugars, lipids, minerals, phenolics and optionally vitamins. In one embodiment the CME comprises 20-80% protein, 10-30% carbohydrates, 5-30% fermentable sugars, 1-10% lipids, 1-10% minerals, and 50-300 μg phenolics per g dry weight.

The term “canola meal” or “CM” as used herein refers to protein meal that is derived from seeds of edible varieties of Brassica napus subsp. Napus. Canola meal can be further characterized, for example, as originating from Brassica napus subsp. Napus seeds comprising limited quantities of erucic acid and/or glucosinolates. For example, canola meal can be protein meal derived from Brassica napus subsp. Napus comprising less than 2% erucic acid and less than 30 μmol of glucosinolates per gram dry weight.

The term “canola meal extract” or “CME” as used herein refers to a mixture of compounds which can include macronutrients or micronutrients, for example proteins, fermentable sugars, lipids, minerals, and vitamins, which are obtained from canola meal through suitable extraction methods. A canola meal extract can, for example, be a liquid extract, a dry extract, for example, wherein all solvents and/or liquid components are evaporated, for example through freeze drying or spray drying. A canola meal extract can, for example, be comprised of a mixture of compounds obtained from a single extraction method, from multiple extraction methods, for example occurring sequentially or in parallel, or from a combination of extraction methods, pretreatment steps, and/or post-treatment modification steps. A canola meal extract can also, for example, be a combination of multiple extracts obtained from canola meal.

The term “protein” as used herein refers to an amino acid or a molecule comprised of multiple amino acid residues, including for example a peptide or a polypeptide, for example a single chain polypeptide, as well as a single chain of a multichain protein, a protein fragment or a full-length protein. Furthermore, as used herein, the term protein can refer to a linear chain of amino acids or it can refer to a chain of amino acids that has been processed and folded into a functional protein. The term protein can also refer to peptides produced by hydrolysis of larger polypeptides.

The term “carbohydrates” as used herein refers to molecules of carbon (C), hydrogen (H) and oxygen (O) atoms. They include sugars, starches, and fiber.

The terms “fermentable sugars” and ‘sugars for fermentation” are used interchangeably herein to refer to sugar molecules, for example mono-, di-, poly-, and oligo-saccharides, that can be used as a carbon source by organisms, for example microbes such as bacteria, fungi, and algae in a fermentation process. Furthermore, as used herein, the term fermentable sugars can refer to sugar molecules that can be converted through fermentation into products such as an alcohol, an acid, a gas, or another desirable product by organisms, for example microbes such as bacteria, fungi, and algae.

In some embodiments, the fermentable sugar is a reducing sugar. The term “reducing sugar” as used herein means a sugar that can act as a reducing agent. Reducing sugars can comprise monosaccharides, disaccharides, oligosaccharides, and some polysaccharides.

The term “lipids” as used herein refers to any of a broad class of hydrophobic or amphiphilic molecules, including for example oils, fats, fatty acids, waxes, mono-, di-, and tri-glycerides, sterols, steroids, fat soluble vitamins and phospholipids or combinations thereof.

The term “minerals” as used herein refers to inorganic substances, for example metals, that are required in the diet of an organism, for example a microbe. For example, a mineral can be iodine, calcium, iron, manganese, potassium, sodium, selenium, chromium, molybdenum, phosphorus, zinc, magnesium, copper or combinations thereof.

The term “vitamins” as used herein refers to organic molecules that are essential micronutrients required in small quantities to support the metabolism of an organism. Vitamins can act as coenzymes and precursors of coenzymes in the regulation of metabolic processes. Vitamins can, for example, include vitamin A, vitamin B1, vitamin B2, vitamin B5, vitamin B6, vitamin B7, vitamin E vitamin K, vitamin C, vitamin D, niacin, pantothenic acid, folic acid, vitamin B12, and derivatives and combinations thereof. The term vitamin as used herein, also means vitamin-like essential nutrients, for example choline.

In one embodiment the CME comprises at least 15% protein by dry weight. In one embodiment the CME comprises at least 20% protein by dry weight. In one embodiment the CME comprises at least 25% protein by dry weight. In one embodiment the CME comprises at least 30% protein by dry weight. In one embodiment the CME comprises at least 35% protein by dry weight. In one embodiment the CME comprises at least 40% protein by dry weight. In one embodiment the CME comprises at least 45% protein by dry weight. In one embodiment the CME comprises at least 50% protein by dry weight. In one embodiment the CME comprises at least 55% protein by dry weight. In one embodiment the CME comprises at least 60% protein by dry weight. In one embodiment the CME comprises at least 65% protein by dry weight. In one embodiment the CME comprises at least 70% protein by dry weight. In one embodiment the CME comprises at least 75% protein by dry weight. In one embodiment the CME comprises at least 50% protein by dry weight In some embodiments, the CME comprises 15-80%, 20-70%, 25-70%, 30-70%, 35-70%, 40-70%, 45-70%, 50-70%, 55-70%, 60-70%, 65-70%, 15-65%, 20-65%, 25-65%, 30-65%, 35-65%, 40-65%, 45-65%, 50-65%, 55-65%, 60-65%, 15-60%, 20-60%, 25-60%, 30-60%, 35-60%, 40-60%, 45-60%, 50-60%, 55-60%, 15-55%, 20-55%, 25-55%, 30-55%, 35-55%, 40-55%, 45-55%, 50-55%, 15-50%, 20-50%, 25-50%, 30-50%, 35-50%, 40-50%, 45-50%, 15-45%, 20-45%, 25-45%, 30-45%, 35-45%, 40-45%, 15-40%, 20-40%, 25-40%, 30-40%, 35-40%, 15-35%, 20-35%, 25-35%, 30-35%, 15-30%, 20-30%, 25-30%, 15-25%, 20-25%, 15-20%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% or 80% by dry weight.

In one embodiment the CME comprises 10-30% carbohydrates by dry weight. In some embodiments, the CME comprises 11-29%, 12-28%, 13-27%, 14-26%, 15-25%, 16-24%, 17-23%, 18-22%, or 19-21% carbohydrates by dry weight. It is noted that the percentage of carbohydrates includes fermentable sugars as described herein.

In one embodiment, the CME comprises at least 5%, 10%, 15%, 20%, 25% or 30% fermentable sugars by dry weight. In some embodiments, the CME comprises 5-35%, 10-35%, 15-35%, 20-35%, 25-35%, 30-35%, 5-30%, 10-30%, 15-30%, 20-30%, 25-30%, 5-25%, 10-25%, 15-25%, 20-25%, 5-20%, 10-20%, 15-20%, 5-15%, 10-15%, 5-10%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% fermentable sugars by dry weight. In one embodiment, the fermentable sugars comprise glucose.

In one embodiment the CME comprises at least 1%, 2%, 3%, 4%, 5%, 6%, 7%, 8%, 9% or 10% lipids by dry weight. In some embodiments, the CME comprises 1-11%, 2-11%, 3-11%, 4-11%, 5-11%, 6-11%, 7-11%, 8-11%, 9-11%, 10-11%, 1-10%, 2-10%, 3-10%, 4-10%, 5-10%, 6-10%, 7-10%, 8-10%, 9-10%, 1-9%, 2-9%, 3-9%, 4-9%, 5-9%, 6-9%, 7-9%, 8-9%, 1-8%, 2-8%, 3-8%, 4-8%, 5-8%, 6-8%, 7-8%, 1-7%, 2-7%, 3-7%, 4-7%, 5-7%, 6-7%, 1-6%, 2-6%, 3-6%, 4-6%, 5-6%, 1-5%, 2-5%, 3-5%, 4-5%, 1-4%, 2-4%, 3-4%, 1-3%, 2-3%, 1-2%, 1%, 2%, 3%, 4%, 5%, 6%, 7%, 8%, 9%, 10%, or 11% lipids by dry weight. In one embodiment, the lipids comprise triacyl glycerides.

In one embodiment the CME comprises 1-10% minerals by dry weight. In one embodiment the CME comprises 1%, 2%, 3%, 4%, 5%, 6%, 7%, 8%, 9% or 10% minerals by dry weight. In one embodiment the CME comprises 2-10%, 3-10%, 4-10%, 5-10%, 6-10%, 7-10%, 8-10%, 9-10%, 4-9%, 5-9%, 6-9%, 7-9%, 8-9%, 4-8%, 5-8%, 6- 8%, 7-8%, 4-7%, 5-7%, 6-7%, 4-6%, 5-6% or 4-5% minerals by dry weight.

In one embodiment, the minerals comprise phosphorus (P), sulfur(S), chlorine (CI), copper (Cu), iron (Fe), manganese (Mn), potassium (K), or calcium (Ca), magnesium (Mg) or zinc (Zn) or combinations thereof. In one embodiment, the minerals comprise 5-25% P. In one embodiment, the minerals comprise 6-30% S. In one embodiment, the minerals comprise 0.01-0.1% CI. In one embodiment, the minerals comprise 2-20% Cu. In one embodiment, the minerals comprise 3-10% Fe. In one embodiment, the minerals comprise 0.01-5% Mn. In one embodiment, the minerals comprise 20-450% K. In one embodiment, the minerals comprise 10-23% Ca.

In one embodiment the CME comprises 50-200 mg of vitamins per kg dry weight. In some embodiments, the CME comprises 50-210, 60-210, 70-210, 80-210, 90-210, 100-210, 110-210, 120-210, 130-210, 140-210, 150-210, 160-210, 170-210, 180-210, 190-210, 200-210, 50-200, 60-200, 70-200, 80-200, 90-200, 100-200, 110-200, 120-200, 130-200, 140-200, 150-200, 160-200, 170-200, 180-200, 190-200, 50-190, 60-190, 70-190, 80-190, 90-190, 100-190, 110-190, 120-190, 130-190, 140-190, 150-190, 160-190, 170-190, 180-190, 50-180, 60-180, 70-180, 80-180, 90-180, 100-180, 110-180, 120-180, 130-180, 140-180, 150-180, 160-180, 170-180, 50-170, 60-170, 70-170, 80-170, 90-170, 100-170, 110-170, 120-170, 130-170, 140-170, 150-170, 160-170, 50-160, 60-160, 70-160, 80-160, 90-160, 100-160, 110-160, 120-160, 130-160, 140-160, 150-160, 50-150, 60-150, 70-150, 80-150, 90-150, 100-150, 110-150, 120-150, 130-150, 140-150, 50-140, 60-140, 70-140, 80-140, 90-140, 100-140, 110-140, 120-140, 130-140, 50-130, 60-130, 70-130, 80-130, 90-130, 100-130, 110-130, 120-130, 50-120, 60-120, 70-120, 80-120, 90-120, 100-120, 110-120, 50-110, 60-110, 70-110, 80-110, 90-110, 100-110, 50-100, 60-100, 70-100, 80-100, 90-100, 50-90, 60-90, 70-90, 80-90, 50-80, 60-80, 70-80, 50-70, 60-70, 50-60, 50, 55, 60, 65, 70, 75, 80, 85, 90, 95, 100, 105, 110, 115, 120, 125, 130, 135, 140, 145, 150, 155, 160, 156, 170, 175, 180, 195, 200, 205, or 210 mg of vitamins per kg dry weight.

In one embodiment, the vitamins comprise vitamin B or choline. In one embodiment, the vitamin B comprises niacin, pantothenate, riboflavin, folic acid, thiamine, biotin, or pyridoxine, or combinations thereof.

Extraction methods can result in the release of antinutrients, for example from lignocellulosic networks, into an extract. The term “antinutrients” as used herein, means a molecule or a compound that interferes with the absorption of nutrients by an organism, for example a microbe. An antinutrient can, for example, inhibit enzymatic activity or prevent mineral uptake through strong binding affinities. Antinutrients include, for example glucosinolates and phenolics. Extraction methods can also result in the release of other undesirable compounds into an extract, for example water-soluble hemicellulose and other impurities, such as ring-based aromatics and nucleic acids. Pretreatment steps, extraction methods, and post-treatment modification steps, for example as disclosed herein and as known in the art, can remove antinutrients and other undesirable molecules and compounds from an extract.

Accordingly, in one embodiment, the CME comprises 50-300 μg phenolics per g dry weight. In one embodiment, the CME comprises less than 300 μg phenolics per g dry weight. In one embodiment, the CME comprises less than 250 μg phenolics per g dry weight. In one embodiment, the CME comprises less than 200 μg phenolics per g dry weight. In one embodiment, the CME comprises less than 150 μg phenolics per g dry weight. In one embodiment, the CME comprises less than 100 μg phenolics per g dry weight. In one embodiment, the CME comprises less than 50-250 μg, 50-200 μg, 50-150 μg or 50-100 μg phenolics per g dry weight. In one embodiment, the phenolic comprises sinapic acid. In one embodiment, the phenolic is sinapic acid.

In another embodiment, the CME further comprises less than 1 μmol glucosinolates per g dry weight. In one embodiment, the CME further comprises less than 0.9, 0.8, 0.7, 0.6, 0.5, 0.4, 0.3, 0.2 or 0.1 μmol glucosinolates per g dry weight.

In another embodiment, the CME further comprises less than 10%, 9%, 8%, 7%, 6%, 5.5%, 5.4%, 5.3%, 5.2%, 5.1%, 5%, 4.9%, 4.8%, 4.7%, 4.6% or 4.5% water-soluble hemicellulose by dry weight.

In another embodiment, the CME further comprises less than 3%, 2.5%, 2.4%, 2.3%, 2.2%, 2.1%, 2.0%, 1.9%, 18%, 1.7%, 1.6% or 1.5% other impurities by dry weight. In another embodiment, the CME further comprises less than 100 μg, less than 90 μg, less than 80 μg, less than 70 μg, less than 60 μg, less than 50 μg, or less than 40 μg other impurities per kg dry weight. In one embodiment the other impurities comprise ring-based aromatics, nucleic acids, or combinations thereof.

In one embodiment, the CME comprises 40-65% protein, 10-30% fermentable sugars, 1-10% lipids and 5-8% minerals by dry weight, and 100-200 mg of vitamins per kg dry weight. In another embodiment, the CME comprises 20-50% protein, 5-25% fermentable sugars, 1-10% lipids and 5-8% minerals by dry weight, and 50-200 mg of vitamins per kg dry weight.

The CME, for example a dry CME, can be combined with a solvent, for example water, for example to improve the bioavailability of its nutrients.

Accordingly, another aspect of the present disclosure is a composition comprising the CME and a solvent.

In an embodiment, the composition is a culture medium supplement. The term “culture medium supplement” as used herein means a supplement or additive that can be added to a culture medium, for example to improve microbial or cell culture growth or survival compared to those cultured in an un-supplemented medium. A culture medium supplement can, for example, contain bioavailable nutrients, for example essential nutrients that are lacking or insufficient in an un-supplemented culture medium or a culture medium not containing a culture medium supplement disclosed herein.

Another aspect of the present disclosure is a culture medium comprising any CME described in the present disclosure or any composition described in the present disclosure.

In one embodiment, the culture medium further comprises one or more additional organic extracts, optionally wherein the one or more organic extracts comprise yeast extract, beef extract, peptone, tryptone, soytone, or a combination thereof. In one embodiment, the one or more organic extracts comprise yeast extract. In one embodiment, the one or more organic extracts comprise beef extract. In one embodiment, the one or more organic extracts comprise peptone. In one embodiment, the one or more organic extracts comprise tryptone. In one embodiment, the one or more organic extracts comprise soytone.

The culture medium can be further supplemented with additional micronutrients or macronutrients. In one embodiment, the culture medium further comprises micronutrients, optionally vitamins, essential minerals, or trace metals; macronutrients, optionally essential amino acids; or combinations thereof. In one embodiment, the culture medium further comprises additional micronutrients. In one embodiment, the culture medium further comprises additional macronutrients. In some embodiments, the culture medium further comprises a combination of additional micronutrients and additional macronutrients. In some embodiments, the additional micronutrients comprise vitamins, minerals, trace metals or combinations thereof. In one embodiment, the additional micronutrients comprise vitamins. In one embodiment, the additional micronutrients comprise minerals. In one embodiment, the additional micronutrients comprise trace metals. In some embodiments, the additional macronutrients comprise amino acids.

The CME, the composition, or culture medium described herein can, for example, have the effect of promoting microbial growth or survival. The term “to promote microbial growth or survival” as used herein, means measurably improving the growth or survival of a microbe. For example, a CME, composition, or culture medium that promotes microbial growth or survival is one that measurably improves the growth or survival of a microbe compared to when the CME, composition, or culture medium is omitted (i.e., compared to a negative control). Measurable improvements can be calculated using any metric known in the art as suitable for measuring the growth or survival of a microbe. Suitable metrics for measuring microbial growth or survival include, for example, an increase in the microbial population, which can be measured, for example, as an increase in number of cells or microbes, an increase in the number of microbe colonies, or an increase in the microbial biomass. Other suitable metrics for measuring microbial growth include, for example, an increase in a metabolic byproduct of the microbe. Measurable improvements in growth or survival can be measured by any means known in the art, for example counting cell/microbe/colony numbers, measuring optical density, for example, using spectrophotometry or colorimetry, weighing dry biomass, taking chromatographic measurements, and running assays including, for example, enzymatic assays.

Accordingly, in some embodiments, the CME, the composition, or the culture medium promotes microbial growth or survival. In one embodiment the CME, the composition, or the culture medium promotes microbial growth. In one embodiment the CME, the composition, or the culture medium promotes microbial survival.

The nutritional profile of canola meal extracts herein disclosed is also suitable for promoting growth or survival of a mammalian cell culture, for example human embryonic kidney (HEK), mouse myeloma (NS0), and Chinese hamster ovary (CHO) cell cultures. Unlike YE, there is little evidence of endotoxins in CME, which are detrimental to mammalian cell lines. The term “to promote growth or survival of a mammalian cell culture” as used herein, means measurably improving the growth or survival of a mammalian cell culture. For example, a CME, composition, or culture medium that promotes growth or survival of a mammalian cell culture is one that measurably improves the growth or survival of a mammalian cell culture compared to when the CME, composition, or culture medium is omitted (i.e., compared to a negative control). Accordingly, in some embodiments, the CME, the composition, or the culture medium promotes the growth or survival of a mammalian cell culture.

The CMEs or compositions herein disclosed can be used as a partial replacement or complete replacement for other extracts, for example costly extracts. Accordingly, in one embodiment is a CME or composition herein disclosed, for use as a partial replacement or complete replacement for an organic extract, optionally yeast extract, beef extract, peptone, tryptone, soytone or a combination thereof. In one embodiment is a CME or composition herein disclosed, for use as a partial replacement or complete replacement for yeast extract, beef extract, peptone, tryptone, soytone or a combination thereof. In one embodiment, the CME or composition is a partial replacement for yeast extract, beef extract, peptone, tryptone, soytone or a combination thereof. In one embodiment, the CME or composition is a complete replacement for yeast extract, beef extract, peptone, tryptone, soytone or a combination thereof. In one embodiment, the CME or composition is a partial replacement for yeast extract. In one embodiment, the CME or composition is a partial replacement for beef extract. In one embodiment, the CME or composition is a partial replacement for peptone. In one embodiment, the CME or composition is a partial replacement for tryptone. In one embodiment, the CME or composition is a partial replacement for soytone. In one embodiment, the CME or composition is a complete replacement for yeast extract. In one embodiment, the CME or composition is a complete replacement for beef extract. In one embodiment, the CME or composition is a complete replacement for peptone. In one embodiment, the CME or composition is a complete replacement for tryptone. In one embodiment, the CME or composition is a complete replacement for soytone.

In another embodiment is a CME or composition herein disclosed, for use as a partial replacement or complete replacement for a nitrogen source. In one embodiment, the CME or composition is a partial replacement a nitrogen source. In another embodiment, the CME or composition is a partial replacement a nitrogen source.

Known in the art are methods for preserving or storing extracts and compositions, for example for ease of handling, for example freeze drying and spray drying methods. Accordingly, in one embodiment, is a CME or composition disclosed herein, wherein the CME or composition is freeze dried or spray dried. In one embodiment, the CME or composition is freeze dried. In one embodiment, the CME or composition is spray dried.

The CME, composition, or culture medium herein disclosed is, for example, food grade. The term “food grade” as used herein, broadly means non-toxic and safe for consumption, for example by livestock, domestic animals, or humans. The term food grade can also refer, for example, to a product with a desirable trait, such as a desirable flavour, aroma, texture, colour, moisture content, cleanliness, density, clarity, protein content, fat content, nutritional profile, condition, or combination thereof. In one embodiment is a CME, composition, or culture medium herein disclosed, wherein the CME, composition, or culture medium is food grade.

### III. Uses and Methods

The CME, composition, or culture medium disclosed herein can be used to culture for example, microbial populations, using methods herein disclosed and known in the art.

Accordingly, another aspect of the present disclosure is use of any CME, composition, or culture medium herein disclosed in a culture with at least one microbe, for promoting microbial growth or survival.

Yet another aspect of the present disclosure is a method for promoting microbial growth or survival, comprising contacting at least one microbe with a CME, composition, or culture medium described herein.

The term “contacting” as used herein means any method known in the art for introducing a microbe into a culture medium in order to promote growth or survival of the microbe, for example inoculating, incubating, culturing, and combining.

Any CME, composition, or culture medium disclosed herein can be used to culture for example, mammalian cell populations, using methods herein disclosed and known in the art. Accordingly, in another embodiment is a use of the CME, composition, or culture medium in a culture with at least one mammalian cell, for promoting growth or survival of the at least one mammalian cell.

Yet another aspect of the present disclosure is a method for promoting growth or survival of at least one mammalian cell, comprising contacting the at least one mammalian cell with a CME, composition, or culture medium described herein.

The CME, composition, or culture medium disclosed herein can be suitable, for example, with a wide array of microbes. In one embodiment, the at least one microbe comprises a bacterium, an archaeum, a fungus, an alga, a protozoan, or a combination thereof. In one embodiment, the microbe comprises a bacterium. In one embodiment, the at least one microbe comprises an archaeum. In one embodiment, the at least one microbe comprises a fungus. In one embodiment, the at least one microbe comprises an alga. In one embodiment, the at least one microbe comprises a protozoan. In one embodiment, the at least one microbe comprises at least two selected from among a group consisting of a bacterium, an archaeum, a fungus, an alga, and a protozoan. In one embodiment, the at least one microbe comprises at least three selected from among a group consisting of a bacterium, an archaeum, a fungus, an alga, and a protozoan. In one embodiment, the at least one microbe comprises at least four selected from among a group consisting of a bacterium, an archaeum, a fungus, an alga, and a protozoan. In one embodiment, the at least one microbe comprises a bacterium, an archaeum, a fungus, an alga, and a protozoan.

In one embodiment, the bacterium is aerobic or anaerobic, or gram-positive or gram-negative. In one embodiment, the bacterium is aerobic or anaerobic. In one embodiment the bacterium is aerobic. In one embodiment, the bacterium is anaerobic. In one embodiment, the bacterium is gram-positive or gram-negative. In one embodiment, the bacterium is gram-positive. In one embodiment, the bacterium is gram-negative. In one embodiment, the bacterium comprises a carboxydotroph, optionally

of genus Cupriavidus, optionally Cupriavidus necator. In one embodiment, the bacterium is of genus Cupriavidus. In one embodiment, the bacterium is Cupriavidus necator. In one embodiment, the carboxydotroph comprises the genus Cupriavidus. In one embodiment, the carboxydotroph comprises the species Cupriavidus necator.

In one embodiment, the bacterium comprises an acetogen, optionally of genus Clostridium, optionally Clostridium carboxidivorans. In one embodiment, the bacterium is of genus Clostridium. In one embodiment, the bacterium is Clostridium carboxidivorans. In one embodiment, the acetogen comprises the genus Clostridium. In one embodiment, the acetogen comprises the species Clostridium carboxidivorans.

In one embodiment, the bacterium produces lactic acid, optionally wherein the bacterium comprises the genus Lactobacillus, Leuconostoc, Pediococcus, Lactococcus, Streptococcus or combinations thereof. In one embodiment, the bacterium comprises the genus Lactobacillus, Leuconostoc, Pediococcus, Lactococcus, Streptococcus or combinations thereof. In one embodiment, the bacterium comprises the genus Lactobacillus. In one embodiment, the Lactobacillus comprises Lactobacillus plantarum, Lactobacillus casei, Lactobacillus rhamnosus, Lactococcus lactis, Lactobacillus acidophilus, Lactobacillus amylophilus, L. bulgaricus, or Lactobacillus helveticus, or combinations thereof. In one embodiment, the bacterium comprises the genus Leuconostoc. In one embodiment, the bacterium comprises the genus Pediococcus. In one embodiment, the bacterium comprises the genus Lactococcus. In one embodiment, the bacterium comprises the genus Streptococcus.

In one embodiment, the bacterium comprises a coliform, optionally of genus Escherichia, optionally Escherichia coli. In one embodiment, the bacterium is of genus Escherichia. In one embodiment, the bacterium is Escherichia coli. In one embodiment, the coliform comprises the genus Escherichia. In one embodiment, the coliform comprises the species Escherichia coli.

In one embodiment, the fungus comprises an oleaginous fungus, optionally of genus Mortierella, optionally Mortierella alpina. In one embodiment, the fungus is of genus Mortierella. In one embodiment, the fungus is Mortierella alpina. In one embodiment, the oleaginous fungus comprises the genus Mortierella. In one embodiment, the oleaginous fungus comprises the species Mortierella alpina.

In one embodiment, the fungus comprises genus Saccharomyces, optionally Saccharomyces cerevisiae. In one embodiment, the fungus comprises Saccharomyces cerevisiae.

In one embodiment, the fungus comprises a saccharifying mold, optionally of genus Aspergillus, optionally Aspergillus oryzae, Aspergillus niger, or a combination thereof. In one embodiment, the fungus comprises Aspergillus oryzae, Aspergillus niger, or a combination thereof. In one embodiment, the fungus comprises Aspergillus oryzae. In one embodiment, the fungus comprises Aspergillus niger.

In some embodiments, is a use or a method described herein for microbial fermentation. The term “microbial fermentation” as used herein is defined broadly as a chemical conversion process carried out one or more microbes.

The CME, composition, or culture medium disclosed herein in culture with at least one microbe can result in the microbe producing a number of products, for example as metabolic byproducts, for example wherein the metabolic byproducts are produced by the bacteria upon metabolizing the CME, composition, or culture medium disclosed herein. These products can, for example, be of further commercial or industrial value, thereby improving the commercial value of CM biomass.

Accordingly, in one embodiment is a use or a method wherein the culture produces polymer, optionally polyhydroxyalkanoates; lipids, optionally polyunsaturated fatty acids, optionally arachidonic acid; proteins, optionally enzymes or recombinant proteins; organic acids, optionally lactic acid; alcohol, optionally ethanol; bacterial carbohydrates, optionally cellulose; fuels, optionally hydrogen; antioxidants; polyphenols; flavonoids; or combinations thereof.

In one embodiment, the culture produces polymer, optionally polyhydroxyalkanoates. In one embodiment is, the culture produces polyhydroxyalkanoates. In one embodiment, the culture produces lipids, optionally polyunsaturated fatty acids, optionally arachidonic acid. In one embodiment is, the culture produces polyunsaturated fatty acids, optionally arachidonic acid. In one embodiment, the culture produces arachidonic acid. In one embodiment, the culture produces proteins, optionally enzymes or recombinant proteins. In one embodiment, the culture produces enzymes or recombinant proteins. In one embodiment, the culture produces enzymes. In one embodiment, the culture produces recombinant proteins. In one embodiment, the culture produces organic acids, optionally lactic acid. In one embodiment, the culture produces lactic acid. In one embodiment, the culture produces alcohol, optionally ethanol. In one embodiment, the culture produces ethanol. In one embodiment, the culture produces bacterial carbohydrates, optionally cellulose. In one embodiment, the culture produces cellulose. In one embodiment, the culture produces fuels, optionally hydrogen. In one embodiment, the culture produces hydrogen. In one embodiment, the culture produces antioxidants. In one embodiment, the culture produces polyphenols. In one embodiment, the culture produces flavonoids.

Another aspect of the present disclosure is a method for culturing at least one microbe with a culture medium disclosed herein for promoting microbial growth or survival, the method comprising inoculating the culture medium with the at least one microbe.

In one embodiment, the method further comprises maintaining the culture at a temperature and pH permissive to microbial growth.

In one embodiment, the method further comprises a first step of preparing the culture medium with a CME or a composition herein disclosed.

A CME or a composition disclosed herein can also be used as a protein supplement for fish. Fish food require a large source of protein, which can be provided, for example by CME. CME can be provided alone to fish, or in a fish food composition comprising additional ingredients. In one embodiment, the fish food composition comprises at least 10%, 20%, 30%, 40%, 50%, 60%, 70%, 80%, or 90% CME.

Accordingly, also provided here in a use of a CME or a composition as described herein for feeding fish. Further provided is a method of feeding fish comprising administering a CME or composition as described herein to the fish. In one embodiment, the fish are hatchlings.

### IV. Methods for Producing a CME

Another aspect of the present disclosure is a method of producing a canola meal extract (CME) comprising:
combining canola meal with a fluid to form a mixture, wherein the mixture has a pH between 3 and 12;
heating the mixture to a temperature between 20° C. and 170° C.; and
extracting the CME from the heated mixture.

In another aspect of the present disclosure is a method of producing a canola meal extract (CME) comprising:
combining canola meal with a fluid to form a mixture,
raising the pH of the mixture to between 9 and 12;
heating the mixture to a temperature between 20° C. and 170° C.;
catalyzing the mixture; and
obtaining the CME from the mixture.

In another aspect of the present disclosure is a method of producing a canola meal extract (CME) comprising:
combining canola meal with a fluid to form a mixture,
heating the mixture to a temperature between 20° C. and 170° C.;
catalyzing the mixture; and
obtaining the CME from the mixture.

The canola meal can be from any source. In one embodiment, the canola meal is finely milled or ground. For example, pelleted canola meal may be ground into a power and then optionally sieved through a filter.

The fluid can, for example, comprise water, optionally deionized water, or another suitable solvent known in the art. The fluid can also comprise one or more of salts, acid and/or bases.

Various ratios of canola meal to fluid are contemplated herein. In one embodiment, the ratio of canola meal to fluid is 0.5-3.15:15-20. In another embodiment, the ratio of canola meal to fluid is 1:5 or 1:10, about 1 to about 5 or about 1 to about 10.

In another embodiment, canola meal is mixed with fluid to maintain a moisture content of 50-80%, optionally 60-70%.

In one embodiment, the mixture of fluid and canola meal is then subject to pH adjustments and/or heating according to the desired method of extraction.

In one embodiment, the extracting comprises water extraction or alkaline extraction.

Alkaline extraction is capable of releasing, for example, proteins and other nutrients from the complex matrix of a cell. Alkaline extraction can also, for example, increase protein solubility by creating a difference between pH and the isoelectric point (PI). Moreover, alkaline extraction also, for example, favors protein hydrolysis. Accordingly, in another embodiment, the extracting comprises alkaline extraction. In one embodiment, the extracting is performed at a pH between 9 and 12, optionally 11 or about 11.

Alkaline extraction is, for example, assisted by increasing temperatures. In one embodiment, the extracting is performed at a temperature between 20° C. and 170° C. In one embodiment, the extracting comprises continuous agitation for 30 minutes to 5 hours.

Accordingly, in one embodiment, the mixture of fluid and canola meal is adjusted to an alkaline pH to allow alkaline extraction. In one embodiment, the pH of the mixture is adjusted to 9-12, 10-12 or 11-12. In another embodiment, the pH of the mixture is adjusted to, between 9-10, 9-11 or 10-11. In another embodiment, the pH of the mixture is, or is adjusted to 8.5, 9, 9.5, 10, 10.5, 11, 11.5, 12 or 12.5.

In one embodiment, the mixture is heated to a temperature between 20° C. and 170° C. In one embodiment, the mixture is heated to at least 20° C., 30° C., 40° C., 50° C., 60° C., 70° C., 80° C., 90° C., 100° C., 110° C., 120° C., 130° C., 140° C., 150, 160° C. or 170° C. In one embodiment, the mixture is heated to 40° C. to 90° C., optionally 50° C. to 75° C. In another, the mixture is heated to 60° C. to 90° C., optionally 70° C. to 80° C. In another embodiment, the mixture is heated to 75° C. or about 75° C., In another embodiment, the mixture is heated to 155° C. to 165° C., optionally 160° C. or about 160° C. In another embodiment, the mixture is heated to 20° C. to 80° C., optionally 25° C. to 75° C.

In some embodiments, the mixture is maintained at the temperature for a period of time. In one embodiment, the mixture is maintained at the temperature for 10 minutes to 2 hours, optionally 20 mins to 1.5 hours, 50 to 70 minutes, 55 to 65 minutes, 1 hour or about 1 hour. In another embodiment, the mixture is maintained at the temperature for 20 to 40 minutes, optionally 25 to 35 minutes, 30 minutes or about 30 minutes.

In some embodiments, the mixture is maintained at the temperature for the under continuous agitation such as continuous stirring.

In some embodiments, the extraction comprises subcritical extraction. Subcritical extraction uses high temperature from 100° C. to 374° C. with pressure enough to maintain fluid in liquid state. Accordingly, in one embodiment, when the mixture is heated to more than 100° C., 155° C. to 165° C., 160° C. or about 160° C., the pressure of the mixture is maintained from 1 to 6 bar.

Alkaline and water based extraction were carried out below 100 degrees, whereas subcritical, either in water (no pH alteration) or in alkaline, was carried out at temperature higher than 100 degree.

In some embodiments, the method of producing a CME herein described comprises a step of catalyzing the mixture. The catalyzing may be performed before or after the extraction steps, namely the steps of raising the pH of the mixture and heating the mixture.

In one embodiment, the catalyzing comprises an enzymatic treatment. In one embodiment, the catalyzing comprises treating the CME with at least one enzyme. In one embodiment, the at least one enzyme comprises a proteolytic enzyme. In one embodiment, the proteolytic enzyme comprises papain, pepsin, alkaline protease, trypsin, Alcalase, or a combination thereof. In one embodiment, the proteolytic enzyme comprises papain. In one embodiment, the proteolytic enzyme comprises pepsin. In one embodiment, the proteolytic enzyme comprises alkaline protease, optionally Alcalase. In one embodiment, the proteolytic enzyme comprises bromelian.

The enzyme is optionally activated prior to treating the CME with the enzyme. Depending on the enzyme used, various methods for activation are contemplated. In some embodiment, the enzyme is activated by incubating in a buffer at pH 3 to 10 and a temperature of 20 to 90° C. for at least 10, 20, 30 40, 50 or 60 minutes. For example, the enzyme papain may be activated in 10 M phosphate buffer with pH 6 to 8 at about 40° C. to 60° C. for at least 20 or 30 minutes.

In one embodiment, 1 part enzyme to 50 to 800 parts dry canola meal, 1 part enzyme to 200 to 300 parts or about 1 part enzyme to 250 parts dry canola meal is added to the mixture.

It has been observed that the pH of the mixture can drop slightly (for example, up to pH 6) as extraction proceeds. Further, after enzymes are added there can be a time dependent decrease of pH (as low as pH 4) as extraction and hydrolysis proceeds. Accordingly, in some embodiments, the pH of the mixture during the enzymatic treatment is adjusted depending on the enzyme used. In one embodiment, the pH of the mixture during enzymatic treatment is adjusted to between pH 3 to 10. In one embodiment, the pH of the mixture during enzymatic treatment with papain is adjusted to between pH 5 to 8, optionally pH 5.5. In another embodiment, the pH of the mixture during enzymatic treatment with alkaline protease is adjusted to between pH 5 to 12.

In one embodiment, the catalyzing comprises treating the CME with the at least one enzyme for between 12 and 96 hours, optionally 24 to 96 hours or 36 to 48 hours, optionally at a temperature between 20° C. and 90° C., optionally between 40° C. to 60° C. In one embodiment, the enzymatic treatment comprises continuous agitation.

In some embodiments, the catalyzing comprises a fungal treatment. In one embodiment, the catalyzing comprises treating the mixture with at least one fungus, optionally of genus Aspergillus, Mortierella, or a combination thereof. In one embodiment, the fungus is Aspergillus oryzae. In another embodiment, the fugus comprises spores. In one embodiment, the mixture is treated with the fungus prior to the extraction steps, namely the steps of raising the pH of the mixture and heating the mixture. In one embodiment, the fungus is incubated with the mixture for between 2 and 7 days, optionally between 24 and 96 hours or about 72 hours. In another embodiment, the fungus is incubated with the mixture at 20° C. to 40° C., 25° C. to 35° C. or about 30° C. Fungus may be applied to the mixture by any method known in the art. In one embodiment, fungal spores are sprayed on the mixture.

In some embodiments, the catalyzing comprises lactic acid fermentation. In one embodiment, the catalyzing comprises incubating the mixture under anaerobic conditions for at least 12, 24, 36, 48, 60, 72, 84 or 96 hours. In another embodiment, the mixture is incubated under anaerobic conditions prior to the extraction steps, namely the steps of raising the pH of the mixture and heating the mixture.

A method of producing a CME herein described may, for example, benefit from a further step of re-extraction. Accordingly, in some embodiments, the method further comprises a step of re-extraction.

A method of producing a CME herein described may, for example benefit from pretreatment steps and/or post-treatment modification steps, for example to remove antinutrients or other impurities from the CM or the CME, to produce fractions concentrated in nutrients that can later be combined to produce a final CME, or to increase the bioavailability of nutrients present within the CME. Accordingly, in some embodiments, the method further comprises at least one pretreatment step, at least one post-treatment modification step, or a combination thereof. In one embodiment, the method further comprises at least one pretreatment step. In one embodiment, the method further comprises at least one post-treatment modification step.

In some embodiments the at least one pretreatment step comprises treating the canola meal with hexane (this is also referred to herein as “defatting”), acidic ethanol, Aspergillus-based pre-digestion or a combination thereof. In one embodiment, the at least one pretreatment step comprises treating the canola meal with hexane. In some embodiments, the at least one pretreatment step comprises treating the canola meal with acidic acid. In one embodiment, the at least one pretreatment step comprises treating the canola meal with Aspergillus-based pre-digestion.

In some embodiments, the least one pretreatment step comprises sterilizing the canola meal. Sterilization may be performed by any method known in the art. In one embodiment, the canola meal is sterilized at high temperature, for example by heating the canola meal to at least 100° C., 110° C. or 120° C. for at least 5, 10 or 15 minutes.

In some embodiments, the at least one post-treatment modification step comprises hydrolysis of macromolecules, optionally carbohydrates or proteins. In some embodiments, the at least one post-treatment modification step comprises hydrolysis of carbohydrates or proteins. In one embodiment, the post-treatment modification step comprises hydrolysis of carbohydrates. In one embodiment, the post-treatment modification step comprises hydrolysis of proteins. In one embodiment, the hydrolysis comprises an acidic treatment, an enzymatic treatment, a microbial treatment, or a combination thereof.

In one embodiment, the hydrolysis comprises an acidic treatment. In one embodiment, the acidic treatment comprises treating the CME with hydrochloric acid, optionally at a pH between 1 and 4, optionally at a temperature between 60° C. and 121° C., optionally for 0.5 to 2 hours. In one embodiment, the acidic acid treatment comprises treating the CME at a pH between 1 and 4. In one embodiment, the acidic acid treatment comprises treating the CME in a temperature between 60° C. and 121° C. In one embodiment, the acidic acid treatment comprises treating the CME for 0.5 to 2 hours.

In one embodiment, the hydrolysis comprises an enzymatic treatment. In one embodiment, the enzymatic treatment comprises treating the CME with at least one enzyme, wherein the at least one enzyme comprises a proteolytic enzyme, a cellulolytic enzyme, or a combination thereof. In one embodiment, the at least one enzymatic treatment comprises a proteolytic enzyme. In one embodiment, the at least one enzymatic treatment comprises a cellulolytic enzyme. In one embodiment, the proteolytic enzyme comprises papain, pepsin, bromelian, or alkaline protease, or a combination thereof. In one embodiment, the proteolytic enzyme comprises papain. In one embodiment, the proteolytic enzyme comprises pepsin. In one embodiment, the proteolytic enzyme comprises alkaline protease. In one embodiment, the at least one enzymatic treatment comprises a cellulolytic enzyme. In one embodiment, the cellulolytic enzyme comprises cellulase, hemicellulose, or a combination thereof. In one embodiment, the cellulolytic enzyme comprises cellulase. In one embodiment, the cellulolytic enzyme comprises hemicellulase. In one embodiment, the enzymatic treatment comprises treating the CME with the at least one enzyme for between 1 and 12 hours, optionally with continuous agitation, optionally at a temperature between 25° C. and 75° C. In one embodiment, the enzymatic treatment comprises continuous agitation. In one embodiment, the enzymatic treatment is at a temperature between 25° C. and 75° C.

In one embodiment, the hydrolysis comprises a microbial treatment. In one embodiment, the microbial treatment comprises treating the CME with at least one fungus, optionally of genus Aspergillus, Mortierella, or a combination thereof, optionally for between 2 and 7 days. In one embodiment, the microbial treatment comprises genus Aspergillus Mortierella, or a combination thereof. In one embodiment, the microbial treatment comprises genus Aspergillus. In one embodiment, microbial treatment comprises genus Mortierella. In one embodiment, the microbial treatment comprises treating the CME for between 2 and 7 days.

In one embodiment, the CME is freeze dried or spray dried. For example, dried powder may be obtained by spray drying the CME.

In some embodiments is a method described herein, wherein the CME produced by the method comprises 20-80% protein, 10-30% carbohydrates 5-30% fermentable sugars, 1-10% lipids, 1-10% minerals, and 50-300 μg phenolics per g dry weight. In some embodiments, the CME produced by the method is a CME as described herein.

In one embodiment, the extracting comprises water extraction and the CME comprises 20-50% protein, 5-25% fermentable sugars, 1-10% lipids, 5-8% minerals, and 50-200 mg vitamins per kg dry weight.

In one embodiment, the extracting comprises water extraction and the CME further comprises less than 100 μg phenolics per g dry weight. In one embodiment, the extracting comprises water extraction and the CME further comprises less than 0.5 μmol glucosinolates per g dry weight. In one embodiment, the extracting comprises water extraction and the CME further comprises less than 50 μg other impurities per kg dry weight.

In one embodiment, the extracting comprises alkaline extraction and the CME comprises 40-65% protein, 10-30% fermentable sugars, 1-10% lipids, 5-8% minerals, and 100-200 mg of vitamins per kg dry weight.

In one embodiment, the extracting comprises alkaline extraction and the CME further comprises less than 300 μg phenolics per g dry weight. In one embodiment, the extracting comprises alkaline extraction and the CME further comprises less than 0.5 μmol glucosinolates per g dry weight. In one embodiment, the extracting comprises alkaline extraction and the CME further comprises less than 5% water-soluble hemicellulose per dry weight. In one embodiment, the extracting comprises alkaline extraction and the CME further comprises less than 2% other impurities per kg dry weight.

### V. Kits

Another aspect of the present disclosure is a kit comprising a CME or a composition herein disclosed, and a culture medium. In an embodiment, the kit further comprises a leaflet containing instructions to produce a supplemented medium.

In one embodiment, the CME or the composition is freeze dried or spray dried. In one embodiment, the CME or the composition is freeze dried. In one embodiment, the CME or the composition is spray dried.

In some embodiments, the kit further comprises one or more organic extracts, optionally wherein the one or more organic extracts comprise yeast extract, beef extract, peptone, tryptone, soytone, or a combination thereof. In one embodiment, the one or more organic extracts comprise yeast extract. In one embodiment, the one or more organic extracts comprise beef extract. In one embodiment, the one or more organic extracts comprise peptone. In one embodiment, the one or more organic extracts comprise tryptone. In one embodiment, the one or more organic extracts comprise soytone.

In some embodiments, the kit further comprises micronutrients, optionally vitamins, essential minerals, or trace metals; macronutrients, optionally essential amino acids; or combinations thereof. In one embodiment, the kit further comprises additional micronutrients. In one embodiment, the kit further comprises additional macronutrients. In some embodiments, the kit further comprises a combination of additional micronutrients and additional macronutrients. In some embodiments, the additional micronutrients comprise vitamins, minerals, trace metals or combinations thereof. In one embodiment, the additional micronutrients comprise vitamins. In one embodiment, the additional micronutrients comprise minerals. In one embodiment, the additional micronutrients comprise trace metals. In some embodiments, the additional macronutrients comprise amino acids.

The following non-limiting Examples are illustrative of the present

### DISCLOSURE

### Example 1—Canola Meal Extracts

Canola meal (CM) comprises a complex network of lignocellulosic biomass wherein protein and other nutrients (non-fiber carbohydrates, vitamins, and minerals) are trapped. To achieve maximum nutritional yield from CM, CM biomass should undergo treatment processes. These treatments can be associated with extreme pH, temperature, or pressure conditions. Post-treatment modification may further lead to beneficial or detrimental modification of the nutrient composition. Increased yield of nutritional components in extracts from CM (CME), along with hydrolyzed protein and carbohydrates in the form of amino acids, peptides, and no-fiber sugars, could be among the beneficial modifications. On the other hand, heat-associated treatment may result in proportional damage to heat-labile amino acids and vitamins (Deleu, 2019). Moreover, there is a need to analyze nutritional composition after each step of pretreatment and extraction to determine which ones produce the most beneficial and the least detrimental modifications to the nutrient composition of CME.

Apart from the nutritional component, specific extraction techniques may release differing amounts of antinutritional phenolic compounds, such as sinapic acid and sinapines, from CM biomass. As alkaline treatment is one of the most practiced methods to release these antinutrients from lignocellulosic networks, a higher amount of sinapic acids and sinapines are reported in known alkaline extracts. These chemicals are reported to have growth inhibiting properties on some microbial species at concentrations under 0.1 g/l (Engles, 2012). Therefore, determining the optimal nutrient extraction techniques, limiting these toxic phytochemicals during the extraction process, and estimating the concentration of these toxic phytochemicals in the final CME product are important considerations. The additional step of phytochemical removal would involve solvent systems which could be associated with a proportional loss of beneficial nutrients. Therefore, comparative studies on extraction techniques, pretreatments, and post-treatments to minimize the antinutritional component while maximizing the nutrition yield in CMEs were undertaken. These techniques and treatments were analyzed, both in isolation and in combination, to validate and optimize extraction techniques to produce optimal CME for microbial culture medium supplementation, for example, wherein the CME is food grade.

### Methods

### Pretreatment of Canola Meal (CM)

Batches of CM were analyzed for pH, moisture, ash content, lipid content, protein content, free amino acids and peptides, carbohydrates, and reducing sugars. Some batches of CM were initially treated with hexane for total extraction of lipid. This step was carried out in a Soxhlet apparatus for 24 hours under a fume hood. Subsequently, the CM was dried and the solvent evaporated, and the defatted CM was then either 1) subjected to additional pretreatment steps prior to CME extraction or 2) proceeded to CME extraction. Additional pretreatment steps included the following, either alone or in combination. Acidic ethanol treatment: treatment with acidic ethanol (EtOH) (30% 1 M HCL and 70% EtOH) was performed to extract soluble phenolics (antinutritional compounds). During phenol extraction, CM to acidic ethanol ratio of 1:10 to 1:30 was maintained for 1 hour at room temperature in an orbital shaker agitated at 100-200 rpm.

Treatment with fungus: fungal (Aspergillus species) based pre-digestion of CM is carried out in solid state culture and/or submerged culture. Sterilized CM (121° C. for 15 min) is spread in a sterile flask, followed by the inoculation with spores/hyphae of fungus (106 Colony Forming Units (CFU) per g of CM) for 3-7 days at 25-35° C. Moisture for this fermentation process is maintained at 55 to 75%.

Extraction of Canola Meal Extract (CME) from CM

A variety of combinations of pH and temperature were evaluated to optimize CME. First, CM was split into 1. CM (without any pretreatment), 2. Defatted CM, and 3. AEOH-DF CM (defatted and treated with acidic EtOH), and Extraction was performed for each category in an alkaline pH range from 9 to 12 at three different temperatures of 25° C., 75° C., and 160° C. Impact of heat on heat-labile proteins was monitored for determining optimal temperatures. Water extraction (without pH treatment thus at a pH range of 6.2-7.2) was also carried out at the above-mentioned temperatures to validate the alkaline extraction results. Extraction was carried out with continuous agitation for 1-5 hours. The sample to solvent ratio ranged from 1:10 to 1:20.

### Digestion and Stabilization of CME (Post-Treatment Modification)

To hydrolyze macromolecules such as proteins and carbohydrates, one or more of the following post treatment modifications are applied. Treatment with hydrochloric acid: treatment of the CME with 4N HCL is done at a pH range of 1 to 4 and at a temperature range of 60° C. to 121° C. for 0.5 to 2 hours. Enzymatic treatment: treatment with one or more proteolytic (protease: pepsin, papain, alkaline protease) and/or cellulolytic (cellulase, hemicellulose) enzymes is done for 2 to 12 hours at temperature ranging from 25-75° C. with continuous agitation. Treatment with fungus: treatment with fungus (Aspergillus, Mortierella) is done for 2 to 7 days with addition of minimal salt and sugar based medium for microbial adaptability.

### Process for Producing a Final CME Product

To obtain a final CME with a desired nutritional profile, extraction pretreatment starts with defatting of CM using hexane, wherein the hexane fraction consisting of oil is discarded. Fungal treatment is then conducted to hydrolyze the biomass. Biomass of CM is used for alkaline extraction at alkaline pH at different temperatures for 1-2 hours, followed by centrifugation. The liquid fraction (supernatant) containing protein, carbohydrates, minerals, and vitamins is hydrolyzed and the pellet containing lignocellulosic and insoluble protein fraction is discarded. Hydrolysis is performed using enzymes at their respective optimal temperatures, pH, and periods. Hydrolysis can also be achieved through acid treatment. After hydrolysis, the product is filtered using 0.45 μm filter and filtrate containing hydrolyzed nutrient is collected, whereas retentate containing biomass impurities and unhydrolyzed nutrient is discarded. Finally, the filtrate is spray dried into a final product.

### Analysis of CME Product

Every experiment is carried out in triplicate. Before and after every pretreatment step, extraction procedure, and post-treatment modification, nutrient concentration in the extract is analyzed, and total recovery is recorded. Results are compared with commercial yeast extract (YE), soytone, and oil seed extracts.

The Kjeldahl method and Bradford assay are used to determine total nitrogen and protein content, respectively. Briefly, the Kjeldahl method comprises the following: to 1 g of sample CME in digestion tube, 7 g of catalyst (9:1 K₂SO₄:CuSO₄) is added followed by 20 ml of Conc. H₂SO₄. Tube is heated at 420° C. in the digestor till color changes to light green and 60 ml of water is added after cooling. 25 ml of 4% boric acid in a conical flask is placed under condenser. The sample is connected to a distillation apparatus with 60 ml of 40% NaOH and heated till ammonia has passed over into boric acid. Conical flask containing ammonia is titrated with 0.1 N H₂SO₄ until the color changes to pinkish.

Briefly, the Bradford assay comprised the following: to a 200 μl sample CME, 1.8 ml Bradford reagent was added. Blank contained 200 μl d.w. & 1.8 ml reagent. After 5 min, absorbance was measured at 595 nm. BSA was taken as standard and concentration Vs absorbance graph was plotted.

The presence of total amino acids is analyzed through the ninhydrin reagent. Briefly, to 1 ml sample CME, 1 ml of ninhydrin reagent is added, vortexed, boiled for 15 min by covering, cooled, and then 5 ml diluent mix (1:1 v/v of water and n-propanol) is added and optical density (OD) at 570 nm is measured. Amino acid standards and glycine are taken as standard and concentration Vs absorbance graph is plotted.

Total reducing sugars and carbohydrates are determined through the 3,5-Dinitrosalicylic acid (DNS) reagent and Anthrone's reagent respectively. Briefly, the DNS reagent method comprised the following: To 1 ml of sample of CME, 3 ml of DNS reagent was added and boiled for 5 min followed by adding of 1 ml sodium potassium tartarate. The cooled sample was analyzed at 510 nm. Glucose was taken as standard and concentration Vs absorbance graph was plotted.

Briefly, Anthrone comprises the following: To 1 ml of sample CME, 5 ml of reagent (2 g anthrone in 1 L conc. H₂SO₄) is added and the sample is incubated at 100° C. for 10 min. Cooled sample is analyzed at 620 nm. Glucose is taken as standard and concentration Vs absorbance graph is plotted.

Macromolecule content is validated through Carbon, Hydrogen, Nitrogen, Sulfur (CHNS) analyzer. Briefly, CME samples are subjected to analyzer and combusted at about 900° C. followed by reduction at about 500° C. Oxygen and helium are used as purge gases. Samples are converted into CO₂, H₂O, N₂, and SO₂ and analyzed accordingly.

Determination of minerals is achieved by X-ray fluorescence spectroscopy (XRF). Briefly, dry CME is converted into ash at 600° C. in a Muffle furnace. At least 1 g of ash content is subjected to XRF analyzer. Condition of XRF includes 20 kV voltage, the X-ray tube with a Pd target, and HighSense silicon drift detector.

Amino acids and vitamins are profiled by high-performance liquid chromatography (HPLC) and their content is validated. Briefly, vitamin measuring methods for water soluble vitamins comprise the following: EC-C18 Poroshell 120 column, 2.7 μm×4.6×100 mm, 0.5 ml/min, ambient, A=0.06% M H₃PO₄ (pH2.5) B=Acetonitrile gradient, DAD@ 205 and 260 nm (BW80 Hz), 20 μl injection.

The concentration of total phenolics is determined by the Folin-Ciocalteu method with trans-sinapic acid as standard. Briefly, 200 μl of appropriately diluted CME is added to 1.9 ml of 10-fold freshly diluted Folin-Ciocateau reagent. After 5 min, 1.9 ml of sodium carbonate solution (60 g/L) is added to the mixture and incubated at room temperature for 90 min. The absorbance of the mixture is measured at 725 nm against a blank. Trans-sinapic acid is used as standard and total phenolic concentration (TPC) is expressed as mg sinapic acid equivalent (SinE)/g (dry basis).

The concentration of other impurities is determined by HPLC analysis. Absorbance range is of 205 nm and 260 nm.

### Freeze Drying and Spray Drying

For storage and handling of extract, it is concentrated into powder through freeze drying or through spray-drying techniques (180° C. inlet air temperature, 1 L/h feeding rate, 35 m³/h air flow rate) and stored as a substrate for fermentation.

Preparing Cupriavidus necator in Culture Supplemented with YE and Various CMEs

Cupriavidus necator in culture was prepared by adding 1:10 ratio of Cupriavidus necator inoculum (48 hours of culture) to media.

Preparing Mortierella alpina in Culture Supplemented with YE and Various CMEs

Mortierella alpina in culture was prepared by adding_100 ml of 7 days M. alpina culture to 1 L media.

Measuring Growth and Polyhydroxy Alkanoate (PHA) Production in Cupriavidus necator in Culture

Briefly, growth was measured as follows: 2 ml of sample of culture in medium after each 3-6 hr of culture was pipetted out in a glass cuvette and optical density was measured at 600 nm. Blank was used as respective media.

Briefly, PHA production was measured as follows: 1 g of freeze-dried cells initially obtained from culture in medium was stirred in 20 mL of chloroform for 48 h at 37° C. followed by a purification process by precipitation with one volume of ice-cold ethanol. Chloroform layer was recovered and dried to measure total content of PHA on dry weight basis.

Measuring Biomass Accumulation and Lipid Production in Mortierella alpina in Shake Flask and Fermenter

Biomass was harvested after 9 days of culture, then centrifuged, washed, and freeze dried. Biomass weight was then measured.

Lipid content was determined by subjecting 1 g of dried biomass in 10 ml of chloroform:methanol:water at 1:1:0.8. Chloroform layer was recovered and dried to measure total content of lipid on dry weight basis.

### Statistics

Statistics were performed using Microsoft Excel. Tukey's Honestly Significant Difference (HSD) test for multiple comparisons at 95% confidence intervals was used to determine statistically significant differences between means. A p-value of less that 0.05 was determined to be statistically significant.

### Results

### Effect of Extraction Techniques on CME Nutritional Profiles

CMEs were prepared using several combinations of pretreatment steps, extraction methods, and post-treatment modification steps as described above. Pretreatment groups included untreated control CM (UT), defatted CM (DF), and acidic ethanol treated and defatted CM (AEOH-DF). Extraction methods included water extraction (WE) and alkaline extraction (AE) methods, and controlled variables included performing the extractions at both 25° C. and 75° C. Total extract (TE), total protein (TP), and total reducing sugar (TRS) were quantified from these extracts.

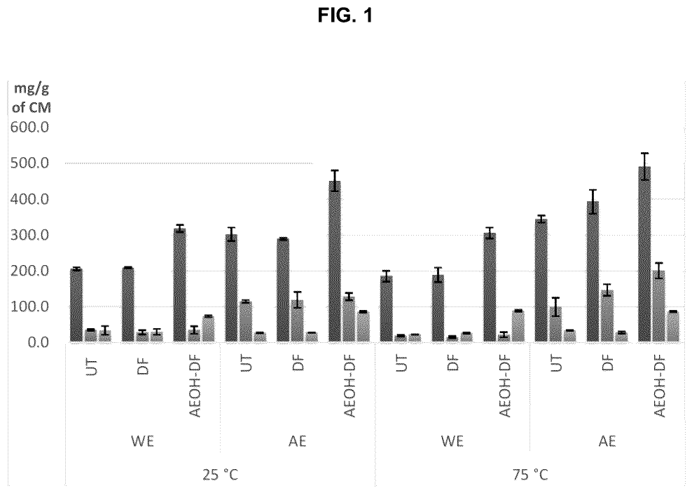

As seen in FIG. 1, the largest total extract, measured as mg per g of CM, was observed for AEOH-DF pretreatments across all extraction methods and temperatures tested. Up to 490 mg of extract was obtained per gram of CM, as seen when AEOH-DF pretreatments were combined with alkaline extraction at 75° C.

Alkaline extraction methods appeared to perform better than water extraction methods across pretreatments and temperatures (FIG. 1). Indeed, total protein (TP) content relative to total extract (TE) was higher in alkaline extracts as compared to extraction in water, reaching levels above 40% of the total extract by mass (Table 1). Absolute mass of total protein was also increased in alkaline extracts compared to extraction in water (Table 1).

TABLE 1
Effects of extraction technique on nutrition extraction from CM.

| | | | T.E. (mg/g | T.P. (mg/g | T.P. | T.R.S. (mg/g | T.R.S. | | | |
|---|---|---|---|---|---|---|---|---|---|---|
| | | | of | of | % of | of | % of | Error | Error | Error |
| | | | CM) | CM) | T.E. | CM) | T.E. | T.E. | T.P. | T.R.S. |
| 25° C. | WE | UT | 205.7 | 34.7 | 16.9 | 33.7 | 16.4 | 4.2 | 2.6 | 12.1 |
| | | DF | 209 | 28.2 | 13.5 | 29.6 | 14.2 | 1.7 | 6 | 8 |
| | | AEOH- | 318.3 | 34.7 | 10.9 | 73.2 | 23 | 10.2 | 10.5 | 2.9 |
| | | DF | | | | | | | | |
| | AE | UT | 302 | 114.7 | 38 | 26.8 | 8.9 | 19 | 3.9 | 0.8 |
| | | DF | 289 | 118.7 | 41.1 | 28 | 9.7 | 3.2 | 22.4 | 0.4 |
| | | AEOH- | 451 | 127.9 | 28.4 | 85.8 | 19 | 29.1 | 10.2 | 2.5 |
| | | DF | | | | | | | | |
| 75° C. | WE | UT | 185 | 19 | 10.3 | 22.7 | 12.3 | 14.9 | 2.6 | 0.4 |
| | | DF | 188.7 | 15.1 | 8 | 26.8 | 14.2 | 20.3 | 2.6 | 1.8 |
| | | AEOH- | 305.3 | 21.6 | 7.1 | 88.7 | 29 | 15.3 | 7.3 | 2.3 |
| | | DF | | | | | | | | |
| | AE | UT | 344.3 | 99 | 28.8 | 33.3 | 9.7 | 9.8 | 25.8 | 1.2 |
| | | DF | 392.7 | 146.2 | 37.2 | 28 | 7.1 | 33.5 | 15.9 | 2.9 |
| | | AEOH- | 490.7 | 200 | 40.8 | 86.3 | 17.6 | 37.2 | 21.6 | 2.2 |
| | | DF | | | | | | | | |

T.E. is total extract;
T.P. is total protein;
T.R.S. is total reducing sugars

Total reducing sugars (TRSs) in the extracts ranged from 7% to 29% of the total extract by mass; however, unlike total protein, TRSs were present in the largest relative mass following water extraction (Table 1). The absolute mass of TRSs was comparable between alkaline extracts and extraction in water (Table 1).

Further, increasing the temperature from 25° C. to 75° C. during extraction appeared to increase total extract yield from CM, particularly in the alkaline extracts (Table 1).

### Effect of CME on Growth and Productivity of Gram-Negative Aerobic Bacteria in Culture

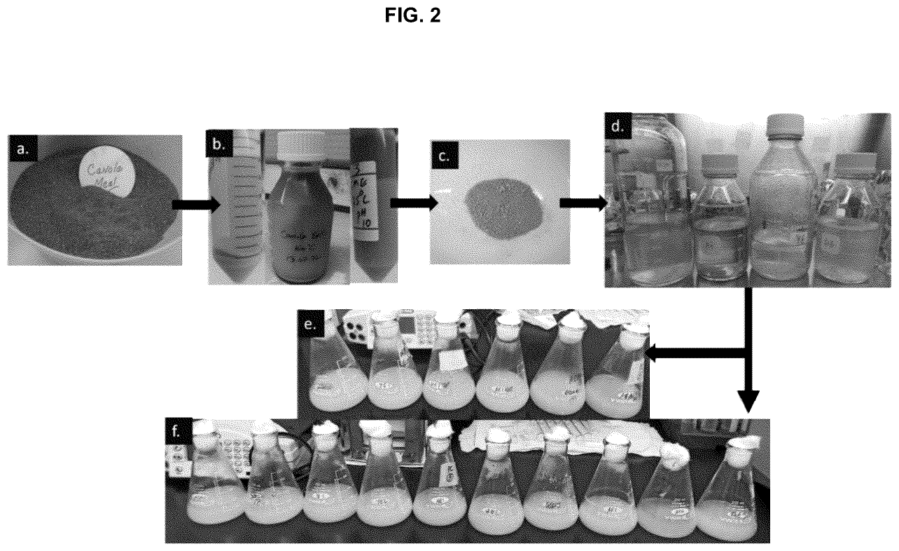

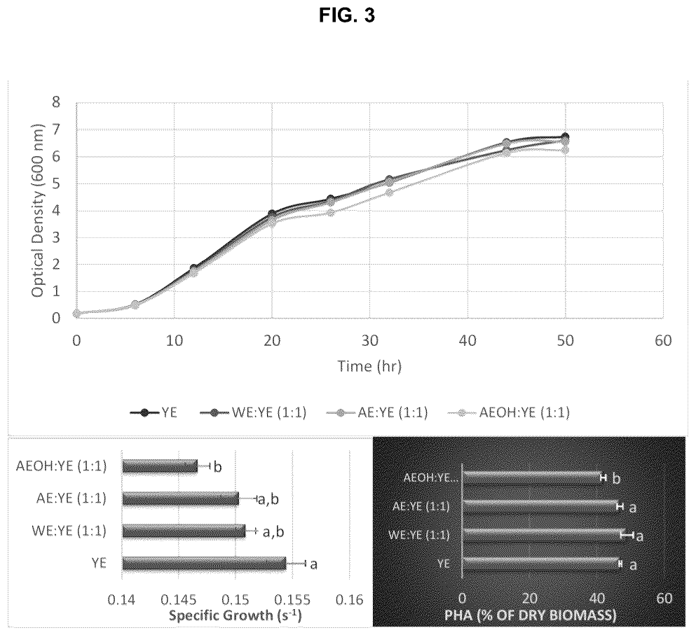

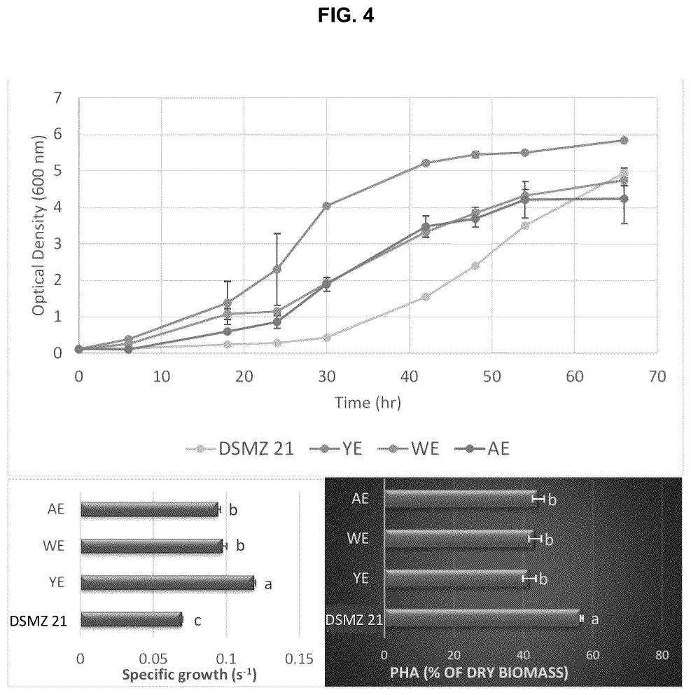

Microbial culture media supplemented with CMEs were prepared as shown in FIG. 2 and as described above, and were used in culture with the gram-negative aerobic bacterium Cupriavidus necator, to evaluate the suitability of various CMEs in promoting growth and survival of gram negative bacteria and of aerobic bacteria. C. necator growth rate and relative polyhydroxy alkanoate (PHA) production were used as measures to compare between media supplements. PHA is a polymer and bioplastic that is an attractive alternative to conventional fossil-based plastic. Unlike other bioplastics, its properties (e.g., thermal transition) are comparable to conventional plastic. Media supplemented with several CMEs described above were compared to media supplemented with the common media supplement, yeast extract (YE), either in combination (i.e., 50% replacement of YE with CME of interest, 1 g total supplement; FIG. 3), or alone (i.e., 100% replacement of YE with CME of interest, 1 g total supplement; FIG. 4).

As seen in FIG. 3, C. necator grown in media wherein the media supplement was 50% YE and 50% CME had similar growth rates (FIG. 3, top panel, and bottom left panel) and produced similar relative amounts of PHA (FIG. 3, bottom right panel) compared to media supplemented with YE-alone. There was no statistically significant difference (wherein statistical significance was defined by a p<0.05) in specific growth rate, or PHA content measured as percent mass of biomass, when 50% of the YE was replaced with an equivalent amount of either water extracted CME (WE:YE) or the alkaline extracted CME (AE:YE). A slight decrease in specific growth rate and PHA content was observed in the media wherein 50% of the YE was replaced with an equivalent amount of a AEOH-DF CME (AEOH:YE) compared to a media supplemented with YE-alone (FIG. 3).

Next, C. necator cultured with DMSZ 21 media wherein YE was completely replaced by CMEs was examined. DSMZ refers to Leibniz Institute DSMZ-German Collection of Microorganisms and Cell Cultures. DSMZ 21 is a special medium formulation with basal chemically defined compounds (minimal salt media) specifically prescribed for certain microbes including C. necator. DSMZ 21 is a basal medium with no organic extracts that was used alone as a negative control. It is alternatively referred to as a minimal salt medium (MSM). As seen in FIG. 4, C. necator grown in media wherein the media supplement was 100% CME (either WE or AE) had a specific growth rate that was statistically significantly higher than DSMZ 21, though statistically significantly lower than when the supplement was 100% YE. Conversely, there was no statistically significant difference in relative PHA production between C. necator grown 100% CME (either WE or AE) vs 100% YE (FIG. 4), demonstrating that CME-alone can support the growth and productivity of gram-negative and aerobic bacteria in culture. All four groups in FIG. 4 contain DSMZ 21. Since, YE and CME are rich in nitrogen and other nutrients, this promoted growth of microbes, whereas DSMZ 21 is deprived of nitrogen, which lead to metabolic response of PHA accumulation.

### Effect of CME on Growth and Productivity of Oleaginous Fungi in Culture

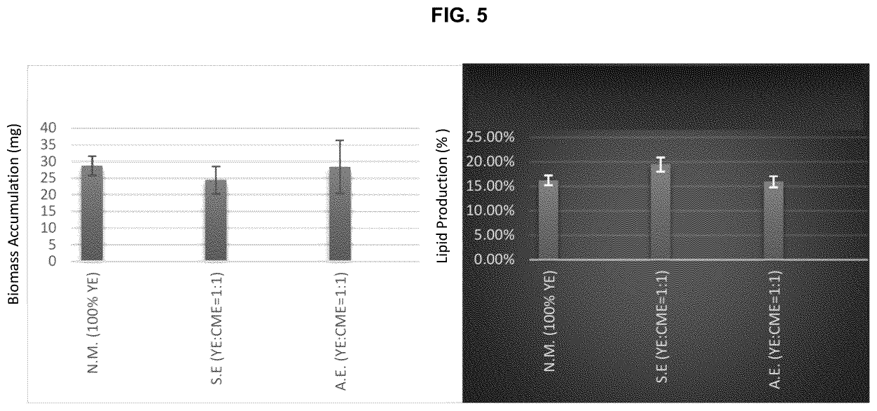

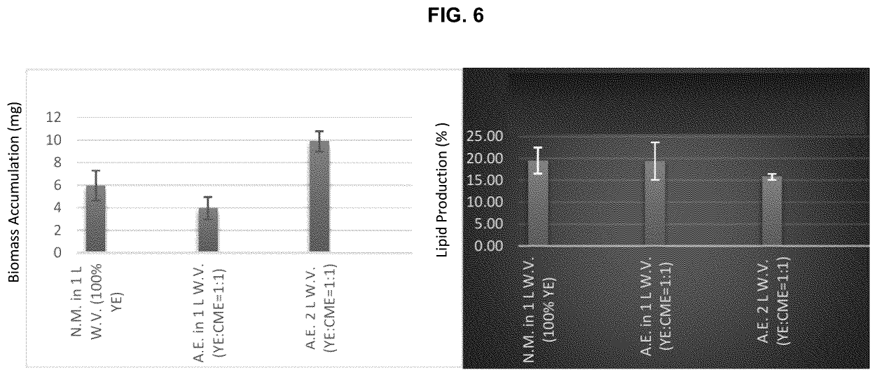

Microbial culture media supplemented with CMEs were prepared as shown in FIG. 2 and as described above, and were used in culture with the fungus Mortierella alpina to evaluate the suitability of various CMEs in promoting growth and survival of fungi, and in particular oleaginous fungi. M. alpina biomass accumulation and relative lipid production were used as measures to compare between media supplements. Lipids produced by M. alpina are rich in arachidonic acid, which can be used, for example in infant milk formula, in chocolates, and also as a nutritional supplement. Normal media supplemented with a combination of CME and YE were compared to normal media supplemented with YE-alone in both shake flask cultures (FIG. 5) and fermenters (FIG. 6). “Normal media” refers to prescribed or reported media with an actual amount of yeast extract and other nutrients being incorporated.

Shake flask cultures were prepared in 50 ml working volume of media supplemented with either 1) 100% YE, 2) 50% YE and 50% CME sub-critically water extracted at 160° C. (S.E. (YE:CME=1:1)), or 3) 50% YE and 50% alkaline extracted CME (A.E. (YE:CME=1:1)). Both biomass accumulation (FIG. 5, left panel) and relative lipid production (FIG. 5, right panel) of M. alpina in culture were similar between the 100% YE supplement and the combined YE:CME supplements, wherein the alkaline extracted CME condition was indistinguishable from YE-alone. There was a slight decrease in biomass accumulation and a slight increase in relative lipid production in sub-critically water extracted CME condition compared to YE, due to lower amounts of organic nitrogen which impairs growth but has a positive effect on lipid accumulation.

Experiments in fermenters were done at both 1 L and 2 L working volumes of media supplemented either with 100% YE or 50% YE and 50% alkaline extracted CME (YE:CME=1:1). 2 L experiments were used as a scale up process and effect of scale up was compared. Compared to 100% YE in 1 L working volume, YE:CME in 1 L working volume had a slight decrease in biomass accumulation (FIG. 6, left panel), but was virtually indistinguishable in relative lipid production (FIG. 6, right panel). Meanwhile doubling the working volume of the YE:CME supplemented culture more than doubled the resultant biomass accumulation, also outproducing the YE condition in 1 L working volume (FIG. 6, left panel). The amount of supplement in the media was proportionally increased with increasing volume. However, doubling the working volume of the YE:CME supplemented culture resulted in a slight decrease in relative lipid production compared to the two conditions in 1 L working volume (FIG. 6, right panel).

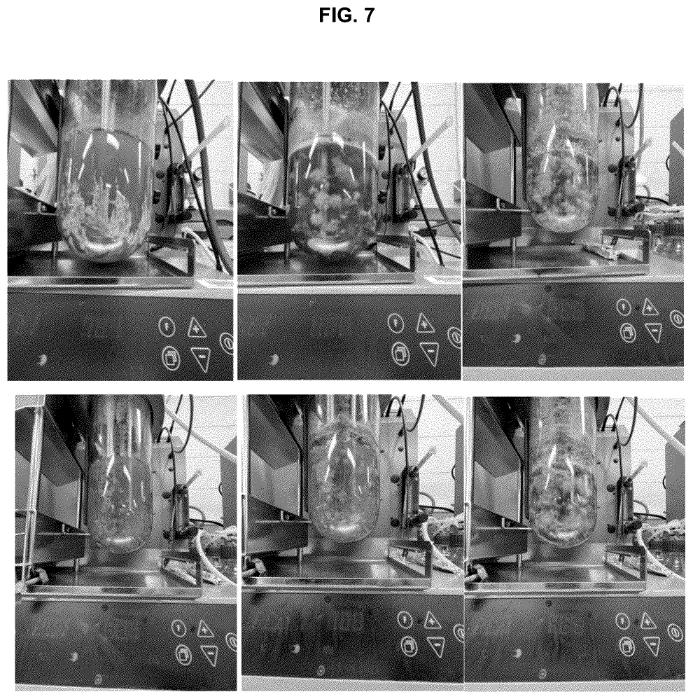

Medium turbidity was assessed for M. alpina grown in culture with 50% YE:CME supplement (FIG. 7, top row) and M. alpina grown in culture with 100% CME supplement (FIG. 7, bottom row). Complex macromolecules such as protein precipitate result in turbid medium, as seen in FIG. 7, left-most panels (day 1 cultures) wherein the medium has a cloudy appearance. Breakdown and consumption of these macromolecules by M. alpina results in a decrease in medium turbidity (FIG. 7, right-most panels; day 7 cultures). While biomass accumulated over time, the clarity of the medium also increased.

These experiments demonstrated that CMEs support both biomass accumulation and relative lipid production in oleaginous fungi cultures.

### Example 2—Preparation of Canola Meal Extract (CME)

To the methods of canola meal (CM) extraction described below, the following pretreatment was applied:
1. Pelleted CM obtained from industries was ground into fine powder;
2. Ground CM was sieved through filter with mesh less than 2 mm;
3. Filtered CM was optionally defatted with hexane through continuous reflux; and
4. Sterilization was done at 121° C. for 15 min. Alternative methods for sterilization could be used.
   Method 1: Extraction of Canola Meal (CM) Using Enzymatic Method Combined with Alkaline Extraction (Optimized Method)

Procedure: Specific to papain. This was also demonstrated with other enzymes like pepsin, trypsin, Alcalase® and combination of these.
1. CM was mixed with deionized water (DW) by maintaining CM to DW ratio 1:10 and added to a reactor.
2. With continuous stirring at 200 rpm pH was raised to 11 with the help of NaOH (up to 6 N) and left for extraction for 1 hour at 50 to 75° C.
3. pH was adjusted below 7.5. This could be a process lead drop due to extracted acids from CM, if not adjustment was performed using HCl (up to 6 N). Note: Depending on enzymes used, pH could be adjusted from 3 to 10.
4. Enzyme was activated in 10 M phosphate buffer with pH 6.8 at 50° C. for at least 30 min before inoculation. Note: Depending on enzymes used, pH temperature could be adjusted from 3 to 10 and 20 to 90° C., respectively.
5. Activated enzymes were inoculated to the reactor. 1 part of enzyme was used for 50 to 800 parts of dry CM.
6. Reaction pH was maintained above 5 at 55° C. and reaction was carried out for 36 to 48 hours with continuous agitation. Note: Depending on enzymes used, pH temperature could be adjusted from 3 to 10 and 20 to 90° C., respectively.
7. Followed by which liquid extract separated using centrifuge at 3000 to 6000 rpm. Dried powder was obtained using spray drier at inlet temperature of 160° C. and outlet temperature below 80° C.

### Method 2: Extraction of Canola Meal (CM) Using Enzymatic Method.

The procedure for this method was like method 1 except step #2 was excluded.

Method 3: Extraction of Canola Meal (CM) Using Aspergillus oryzae Followed by Alkaline Extraction

### Procedure:

1. DW was added to CM to maintain 60-70% moisture content and mixed uniformly.
2. 10⁷ colony forming units (CFU) of A. oryzae spores were uniformly sprayed on moist CM and incubated at 30° C. for 72 hours.
3. Fermented CM was mixed with deionized water (DW) by maintaining CM to DW ratio 1:10 and added to a reactor.
4. With continuous stirring at 200 rpm pH was raised to 11 with the help of NaOH (up to 6 N) and left for extraction for 1 hour at 75° C.
5. Followed by which liquid extract separated using centrifuge at 3000 to 6000 rpm. Dried powder was obtained using a spray drier at inlet temperature of 160° C. and outlet temperature below 80° C.
   Method 4: Extraction of Canola Meal (CM) Using Aspergillus oryzae.

The procedure for this method was like method 3 while in step #4 no alteration of pH was done.

### Method 5: Extraction of Canola Meal (CM) Using Lactic Acid Fermentation Followed by Alkaline Extraction.

### Procedure:

1. Un-sterilized CM was mixed with deionized water (DW) by maintaining CM to DW ratio 1:10 and added to a reactor.
2. This was left overnight covered with muslin cloth after which anaerobic condition was maintained up to 96 hours for lactic acid fermentation.
3. With continuous stirring at 200 rpm pH was raised to 11 with the help of NaOH (up to 6 N) and left for extraction for 1 hour at 75° C.
4. Followed by which liquid extract separated using centrifuge at 3000 to 6000 rpm. Dried powder was obtained using a spray drier at inlet temperature of 160° C. and outlet temperature below 80° C.

### Method 6: Extraction of Canola Meal (CM) Using Lactic Acid Fermentation Followed by Water Extraction.

The procedure for this method was like method 5 while in step #3 no alteration of pH was done.

### Method 7: Alkaline Extraction of Canola Meal (CM).

### Procedure:

1. CM was mixed with deionized water (DW) by maintaining CM to DW ratio 1:10 and added to a reactor.
2. With continuous stirring at 200 rpm pH was raised to 11 with the help of NaOH (up to 6 N) and left for extraction for 1 hour at 25° C. to 75° C.
3. Followed by which liquid extract separated using centrifuge at 3000 to 6000 rpm. Dried powder was obtained using a spray drier at inlet temperature of 160° C. and outlet temperature below 80° C.

### Method 8: Water Based Extraction of Canola Meal (CM).

The procedure for this method was like method 7 while in step #2 no alteration of pH was done.

### Method 9: Subcritical-Alkaline Extraction of Canola Meal (CM).

### Procedure:

1. Un-sterilized CM was mixed with deionized water (DW) by maintaining CM to DW ratio 1:10.
2. With continuous stirring at 200 rpm pH was raised to 11 with the help of NaOH (up to 6 N).
3. Mixture was poured in a reactor vessel and extracted at 160° C. for 30 minutes followed by its cooling cycle to 25° C.
4. Followed by which liquid extract separated using centrifuge at 3000 to 6000 rpm. Dried powder was obtained using a spray drier at inlet temperature of 160° C. and outlet temperature below 80° C.

### Method 10: Water Based Sub-Critical Extraction of Canola Meal (CM).

The procedure for this method was like method 9 while in step #2 no alteration of pH was done.

### Analysis of Biomolecules

To assess protein and total nitrogen content, the Bradford assay and CHNS elemental analysis was conducted (Bradford, 1976; Mandal et al., 2017). The content of protein hydrolysates was analyzed through the ninhydrin method (Smith & Agiza, 1951). Content of reducing sugars was determined through the DNS method (Miller, 1959). PHA content was analyzed after choloroform extraction as reported by Law & Slepecky (1961). Total carbohydrate was analyzed through Anthrone's method. Elemental minerals were analyzed through X-ray fluorescence (XRF) analyzed as percentage of CME ash. Presence of vitamins like niacin, riboflavin, thiamine, biotin, and pyridoxine. were analyzed through HPLC analysis (Method for water soluble vitamins, EC-C18 Poroshell 120 column, 2.7 μm×4.6×100 mm, 0.5 ml/min, ambient, A=0.06% M H₃PO₄ (pH2.5) B=Acetonitrile gradient, DAD@ 205 and 260 nm (BW80 Hz), 20 μl injection) and, pantothenate and choline were analyzed through ion chromatography (Method for water soluble vitamins, AS11-HC-4 μm column, 4 μm×4 mm×250 mm, 1 ml/min, ambient, 20 mM KOH, CD, 1.5 ml injection).

### Results

Table 2 shows the total contents of extracts and percentages of nutrients in the extracts. Overall, it is shown that method 1 has high yield both in terms of extract and protein content in it. The % of carbs includes reducing sugars. Reducing sugars are derived from carbohydrates; for example they can be monomers, dimers, or oligomers of starch/carbohydrates.

TABLE 2
Total contents from different extraction methods

| | | Extract | | | | | | Phenolics |
|---|---|---|---|---|---|---|---|---|
| | Pdt. | (mg/g of | | | Reducing | | | (mg/g of |
| Methods | Name | CM) | Protein | Carb. | sugar | Lipids | Minerals | Ext.) |
| 1 | AE_P | 419.67 ± 57 | 77.9 ± 5% | 14.2 ± 3% | 7.6 ± 3% | 3.2 ± 0.5% | 7.3 ± 0.3% | 0.17 ± 0.07 |
| 2 | WE_P | 399.07 ± 31 | 70.8 ± 6% | 17.9 ± 6% | 7.3 ± 3% | 2.7 ± .07% | 5.6 ± 0.7% | 0.19 ± 0.03 |
| 3 | Ao_AE | 232.93 ± 40 | 59.9 ± 3% | 23.6 ± 8% | 17.0 ± 2% | 8.2 ± 1.0% | 3.7 ± 0.3% | 0.21 ± 0.04 |
| 4 | Ao_WE | 267.40 ± 27 | 49.8 ± 7% | 26.1 ± 5% | 19.4 ± 5% | 9.8 ± 2.1% | 4.5 ± 0.7% | 0.19 ± 0.01 |
| 5 | LB_AE | 364.30 ± 35 | 54.1 ± 9% | 23.1 ± 7% | 09.2 ± 2% | 12.4 ± 3.6% | 5.2 ± 1.8% | 0.17 ± 0.04 |
| 6 | LB WE | 224.80 ± 22 | 33.4 ± 5% | 26.1 ± 4% | 12.2 ± 3% | 9.3 ± 1.6% | 3.9 ± 0.6% | 0.19 ± 0.06 |
| 7 | AE | 195.50 ± 12 | 40.0 ± 4% | 13.3 ± 4% | 08.0 ± 2% | 5.3 ± 1.8% | 5.2 ± 2.3% | 0.11 ± 0.02 |
| 8 | WE | 104.50 ± 26 | 28.5 ± 3% | 23.0 ± 7% | 14.4 ± 2% | 3.8 ± 1.1% | 5.1 ± 1.1% | 0.09 ± 0.01 |
| 9 | SC_AE | 288.72 ± 71 | 51.8 ± 8% | 21.5 ± 3% | 12.4 ± 3% | 6.1 ± 2% | 9.0 ± 1.9% | 0.27 ± 0.03 |
| 10 | SC_WE | 346.37 ± 38 | 43.8 ± 6% | 26.3 ± 6% | 13.2 ± 5% | 4.4 ± .7% | 8.2 ± 3.1% | 0.23 ± 0.07 |

Table 3 shows the contents of elements detected in the minerals. Elements like Mg, Cu, and Zn could also be present in the extract.

TABLE 3
Elements percentage of total minerals.

| P | S | Cu | Fe | Mn | K | Ca |
|---|---|---|---|---|---|---|
| (%) | (%) | (%) | (%) | (%) | (%) | (%) |
| 5 to | 6 to | 2 to | 3 to | 0.1 | 20 | 10 |
| 25% | 30% | 20% | 10% | to 5 | to | to |
| | | | | % | 45% | 30% |

Presence of vitamins were confirmed in the range of 50 to 500 mg. Peaks for niacin, riboflavin, thiamine, biotin, pyridoxine, pantothenate and choline were observed whereas folic acid and vitamin E were not resolved from the method.

TABLE 4
Properties of CME.

| | | Extract |
|---|---|---|
| | | precipitation on |
| Extraction type | Solubility | sterilization |
| Alkaline Extract | Partial | High |
| Water Extract | Partial | Medium |
| Subcritical Extract | Medium | Medium |
| Enzyme Extract | High | Low |
| A. oryzae Extract | High | Low |
| Lactic acid Extract | High | Low |

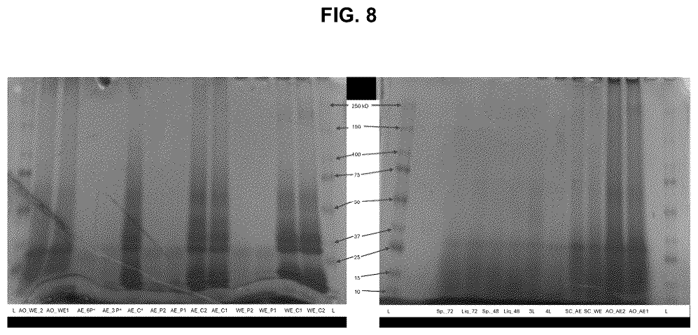

FIG. 8 shows the molecular weight of proteins present in the extracts. There were high molecular weight proteins in water and alkaline extracted canola meal and protein fractions were seen between 250 and 150 kDa, and proteins were more prominent between 75 and 10 kDa. A diminishing high molecular protein band was seen in A. oryzae catalysis, this was more focused on cleaving proteins sized between 250 to 37 kDa and subcritical cleaved. Although results for subcritical extracts were similar to A. oryzae extracts, bands were more diminished here with prominent bands between 37 and 25 kDa and this shows the ability of high temperature to hydrolyze proteins. Moreover, results from papain-based extractions were promising with most of the proteins being hydrolyzed and faint remains of protein between 37 to 25 kDa.

### Effect of CME on Microbial Growth and Product Yield

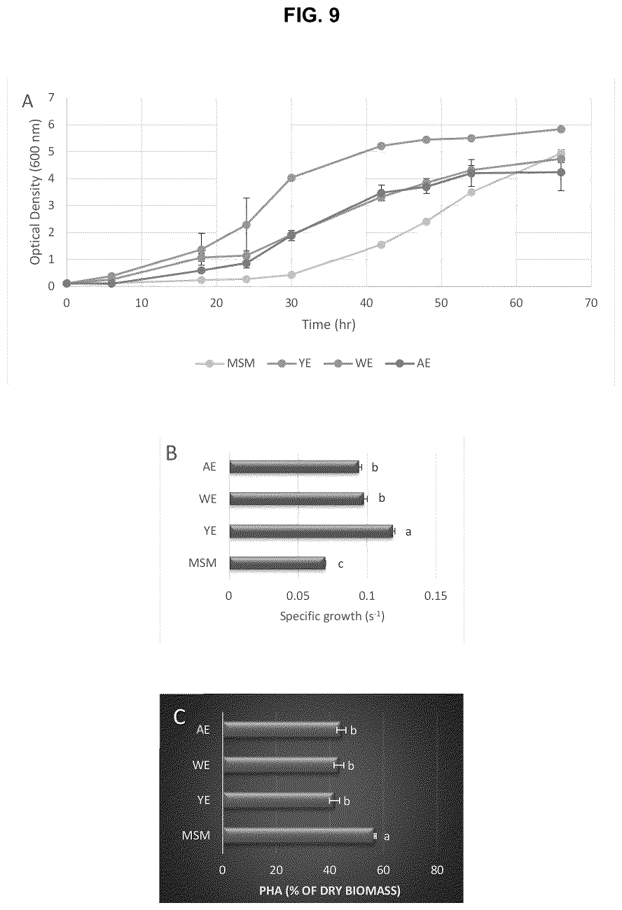

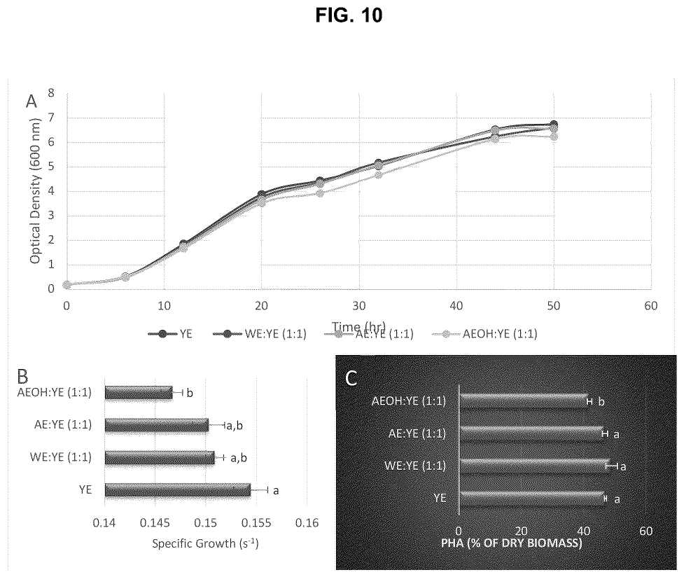

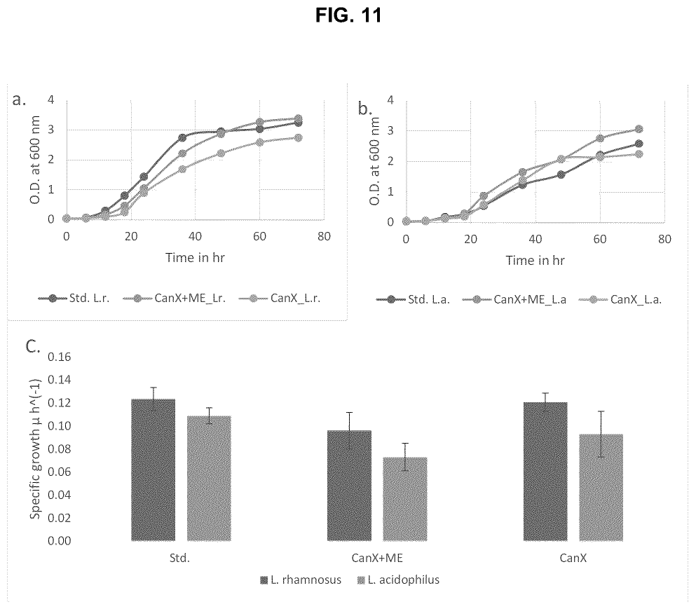

FIG. 9 and FIG. 10 show the growth rates of gram-negative aerobic microbe at 100% and 50% replacement of YE with CME and effect of this in bioplastic (PHA) production. There is no significant difference (p<0.05) in specific growth rate and PHA content in biomass when YE is replaced CME at 50% and this was true for water extracted at normal pH (WE) and alkaline extract (AE). A slight decrease of specific growth rate and PHA content was observed in df-AcEtOH extract. Without being bound by theory, this could be because of high salt content released after neutralization of acidic treatment. Further, growth of bacteria at 100% replacement of YE with CME and effect of this in bioplastic (PHA) production was compared with a basal media with no organic extracts. Although, specific growth of CME was lower than YE supplement, this growth was significantly higher than minimal salt media (MSM). Proteins in YE undergoes autohydrolysis by their indigenous enzymes before they are presented as final product (SigmaAldrich, n.d.). There was no significant difference in the PHA content in biomass in the YE and CME supplemented media. This shows CME alone can support the growth and productivity of microbes.

While the results above indicated that alkaline extracts from df-AcEtOH have a good nutrition profile with higher total extract, amino acids/peptides, and reducing sugars, microbial growth and productivity in this extract was seen to be comparatively less (FIG. 10). Without being bound by theory, neutralization steps and removal of salt accumulated through neutralization could optimize its quality.

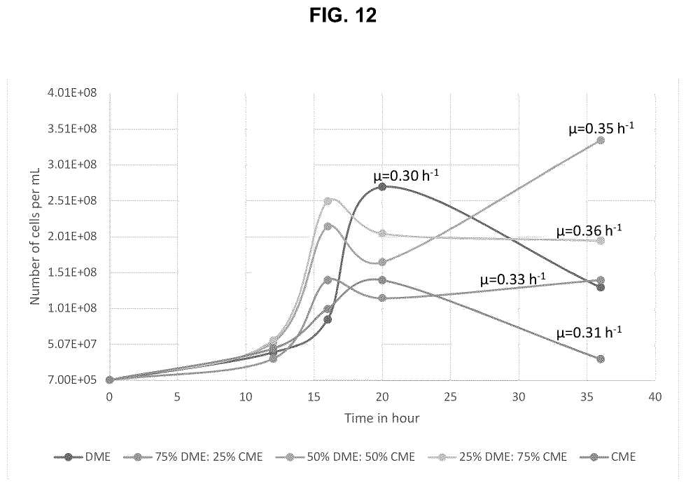

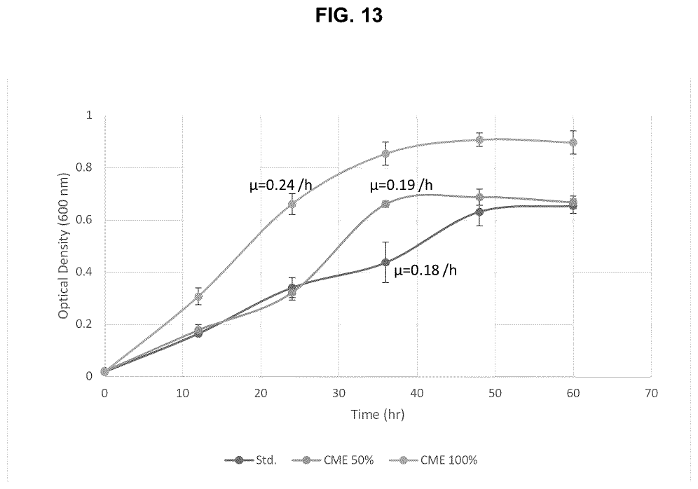

In FIG. 12, no difference in specific growth compared to standard MRS media when YE and ME were replaced by papain catalyzed CME was seen. Whereas growth rate was lower in both L. rhamnosus and L. acidophilus when only yeast extract was replaced keeping meat extract intact. The rate of cell accumulation in Saccharomyces cerevisiae when supplemented with CME was higher as compared to media supplemented with dry malt extract (DME) only (FIG. 12). These results show that CME can be used to enhance growth of anaerobic microbes. Moreover, in Clostridium carboxidivorans species there is a higher growth rate in CME as compared to YE (FIG. 13). Cells like Clostridium, Saccharomyces, and Lactobacillus are either strict anaerobes or facultative anaerobes, along with good nitrogen content high antioxidant activity of CME could support activity of these cells and these results were supported by a DPPH assay.

Utilization of CME by Mortierella alpina for Arachidonic Acid Production

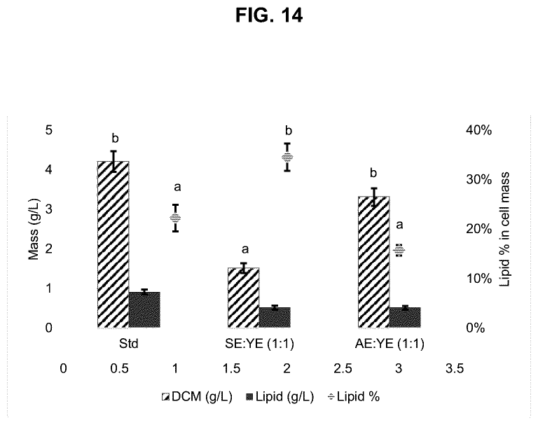

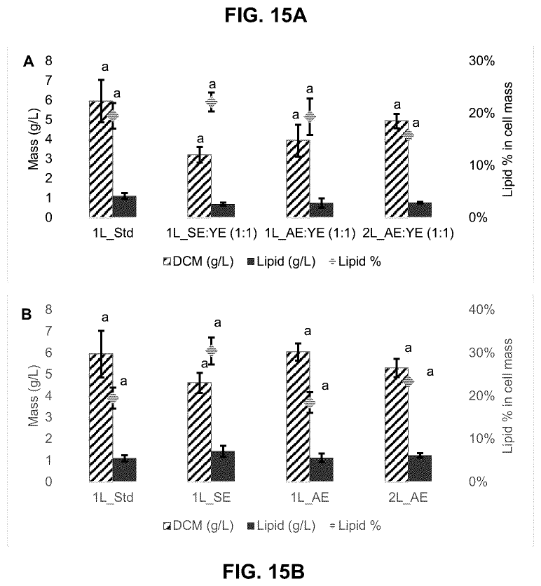

As an alternative to YE for M. alpina CME was investigated with 50% and 100% replacement of YE with CME (FIGS. 14 and 15). Growth of fungus was supported by 50% CME in media; however, biomass accumulation was visibly high in standard media. In the shake flask culture, biomass yield of SE:YE (1:1) was significantly lower (P<0.05) in shake flasks as compared to other two media types, whereas lipid content of this media was highest of 34.5% of DCM (FIG. 14).

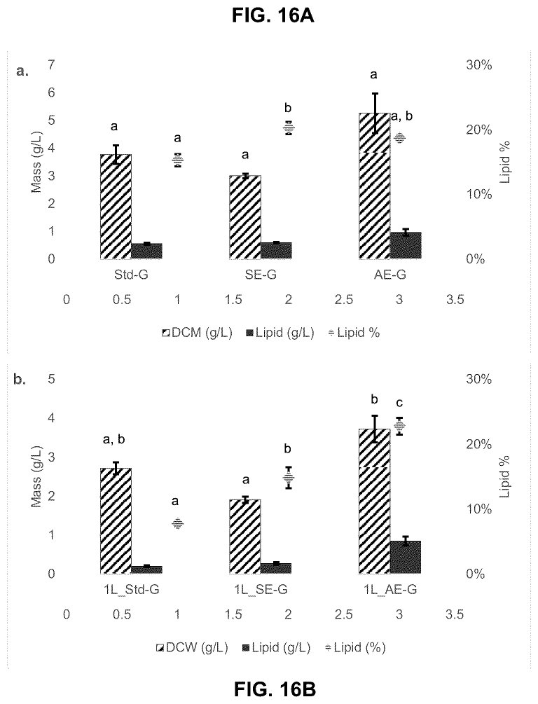

Scaled-up cultures in fermenters resulted in higher biomass yield as compared to shake flasks as fermenters were continuously aerated. Although observed dry cell mass (DCM) and lipid accumulation for fungus grown in the media replaced by 50% CME in 1- and 2-L fermenters was lower than standard YE media, there was no statistical difference (P<0.05) between them (FIG. 15A). Similar statistics revealed no significant difference between means of growth and lipid content was obtained for 100% replacement of YE with CME in the fermenters. Observed DCM and lipid obtained from AE in 1-L fermenters were comparable to that of the standard media and these were up to 6 g/L and 19% of DCM, respectively (FIG. 15B.). Although SE accumulated comparatively less biomass, lipid composition in biomass when grown on this medium was as high as 30%. However, ANOVA result specifies that there were no significant differences (P<0.05) between means during this experimental run for 100% replacement of YE by CME.

Where external glucose supply was eliminated in all three media, i.e., standard media-G, SE-G, and AE-G, biomass yield and lipid content was greatest in the AE-G formulation (FIG. 16). A similar trend was observed during both shake flask (FIG. 16A) and scale-up culture in fermenters (FIG. 16B). The lowest biomass mean was seen in SE-G culture; whereas, average lipid content was lowest in standard media without glucose. Although growth did not differ significantly in shake flask culture, lipid content was significantly higher (P<0.05) in the biomass resulted from AE-G culture for both studies. When comparing the scale-up cultures, removing glucose significantly (P<0.05) affected biomass accumulation. The greatest biomass was obtained in glucose 1 L_AE-G was 3.7 g/L which was about 37% less than that of 1 L_AE culture.

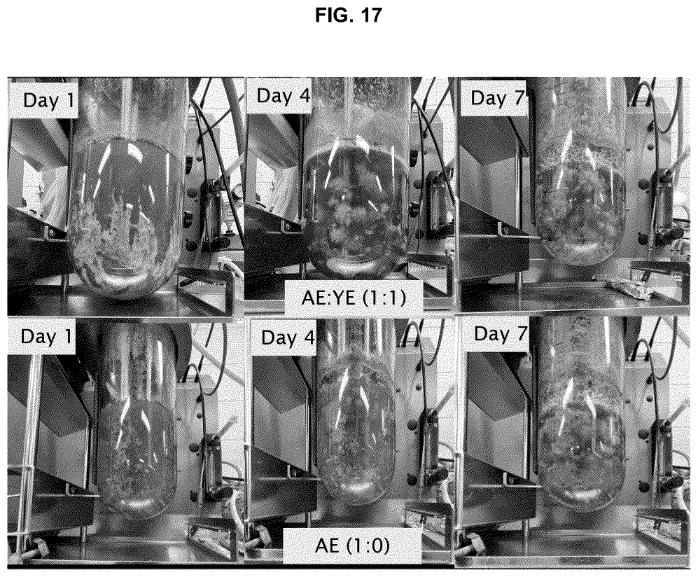

Replacement of YE by CME provided some unexpected results. At 50% replacement of YE in the medium, standard media significantly increased the fungal biomass as compared to media with CME, but differences in biomass yield with 100% YE replacement were not significant. However, M. alpina growth in standard media with YE was observed to be comparatively stable and at the higher end in all these studies except for glucose-minus trials. Commercial YE as medium component undergoes autohydrolysis by its inherent proteolytic enzymes, thus hydrolyzed protein should be readily accessible for microbial uptake (Tao et al., 2023). This study exerted minimal effort towards protein hydrolysis, with any observed hydrolysis likely occurring during the extraction process. One of the interesting observations while supplementing CME in M. alpina culture was that there was significant change in culture appearance with time (FIG. 17). Initially, sterilized CME media with precipitated proteins along with fibrous carbohydrates tends to develop haze and increase turbidity of the medium. After culture progresses with time, drastic increase in mycelial clumps was visualized with disappearance of haze and precipitated proteins. Lahn et al. (2002) observed that enzymes activities associated with breakdown of complex macromolecules, such as endo-cellulase, endo-chitinase, β-1, 3-glucanase and protease, were released in the medium by M. alpina and proteolytic activities were highest among these. Similarly, M. alpina was reviewed as plant growth promoter by relating its ability to breakdown complex carbohydrates in the soil (Ozimek & Hanaka, 2021).

Results for lower biomass and higher lipid accumulation for SE yielded interesting outcomes. The most investigated cause of reduced biomass and elevated lipid accumulation in oleaginous microorganisms is nutrition starvation, in particular, nitrogen limitation (Lv et al., 2021; Yaakob et al., 2021). Deficient nitrogen results in down-regulation of isocitrate dehydrogenase levels in the citric acid cycle which in turn directs glycolytic carbon towards fatty acid synthesis, a metabolic shift that previously has been verified in M. alpina (Lu et al., 2020). In this study, nitrogen percent in SE was higher (0.68 g/L, shown in Table 5) as compared to other media types, but nitrogen utilization by M. alpina was very low as compared to AE and standard media. Table 5 shows the utilization of carbon (C) and nitrogen (N) from the culture media by M. alpina and provides C—N balance and total mass balance of the culture. Only about 37% and 18% of total supplemented nitrogen was utilized by the fungus cultured in 1 L fermenter consisting of SE media with and without glucose, respectively. On the other hand, nitrogen accumulation was as high as 87% and 57% with AE plus and minus glucose, respectively. Further, nitrogen accumulation could also be supported by consumption of carbon sources in the media, as analyzed by changes in C/N ratio between media, biomass, and effluent (see Table 5). The difference in C/N ratio between culture effluent and media were positive for when glucose was included culture and negative for when glucose was excluded (except for AE-G). Moreover, this difference in SE-supplemented culture was as high as 83% and this is consistent with low carbon consumption eventually leading to lower biomass accumulation. These results indicate that low nutrient uptake in SE extract can be related to heat treatment during subcritical extraction which could denature protein structure and destruction of heat sensitive amino acids. This change in protein could limit the interaction between the target functional group in the polypeptide chain and specific binding site of the proteolytic enzymes, thus affecting the availability of nitrogen source in the culture (Leite et al., 2021). Moreover, amino acids as nitrogen source has been reported to effect transcriptional controls like nitrogen catabolite repression control (NCR) and general control of amino acid biosynthesis (GAAC), and could be related with growth and lipid production in Saccharomyces cerevisiae (Godard et al., 2007).

Two predominant proteins, cruciferin and napin in canola meal has distinct structural profile which defines their physiochemical properties (Perera et al., 2016). Napin contains relatively short chain polypeptides with dominant a helical structure and is more soluble and stable in wide range of pH and temperature. On the hand, cruciferin has long polypeptide chain, where β-sheet is dominant and prone to denaturation with shifts in pH and temperature. Further study on effect of subcritical treatment on protein structure and amino acid profile of canola meal and the effect these changes have on M. alpina growth and productivity could clarify ambiguities related to nitrogen content and its utilization from extracts of canola meal. In standard media, nitrogen utilization by M. alpina was about 49% for both the media with and without glucose, and this was relatively lower than seen for AE. This result supports the findings of Higashiyama et al. (1998) who reported preference of nitrogen source to increase growth and ARA productivity of M. alpina was soy flour with unmarred protein as compared to YE, when there were minerals in the media. Alkaline extract of canola meal was found to lead to better biomass yield and increase ARA content in the biomass (Table 7).

During the studies with M. alpina batch-wise variation was found in the biomass accumulation by the fungus even though similar operation conditions were applied. For all treatments used during this research, including shake flask culture, aerated flask culture, culture in fermenters, and cultures with or without CME, these variations were prevalent. Reasons for such experimental variations are unknown. During our studies with 50% and 100% replacement of alkaline extract of CME, we observed fungal responded well in 100% YE replacement (AE) as compared to 50% replacement (AE:YE (1:1)). This deviated with the hypothesis of the study which forecast that the biomass accumulation with AE:YE (1:1) should be near that of standard media as compared to AE.

TABLE 5
Carbon (C) and Nitrogen (N) profile M. alpina cultures, and their
elemental and total mass balance.

| Media | Std. | AE | SE | Std.-G | AE-G | SE-G |
|---|---|---|---|---|---|---|
| type* | (g/L) | (g/L) | (g/L) | (g/L) | (g/L) | (g/L) |
| a. Dry | 59.6 | 64.6 | 64.6 | 9.6 | 14.6 | 14.6 |
| mass of | | | | | | |
| medium | | | | | | |
| C | 21.90 | 23.97 | 24.20 | 1.91 | 3.97 | 4.20 |
| N | 0.64 | 0.50 | 0.68 | 0.64 | 0.50 | 0.68 |
| b. C/N | 34.22 | 48.04 | 35.43 | 2.98 | 7.96 | 6.15 |
| c. Total | 22.54 | 24.47 | 24.88 | 2.56 | 4.47 | 4.88 |
| (C + N) | | | | | | |
| d. Dry cell | 5.95 | 6.04 | 4.61 | 2.72 | 3.73 | 1.91 |
| mass | | | | | | |
| C | 1.51 | 2.47 | 1.31 | 1.21 | 1.49 | 0.78 |
| N | 0.31 | 0.43 | 0.25 | 0.31 | 0.28 | 0.12 |
| e. C/N | 4.82 | 5.70 | 5.17 | 3.85 | 5.27 | 6.30 |
| f. Total | 1.83 | 2.91 | 1.56 | 1.52 | 1.77 | 0.90 |
| (C + N) | | | | | | |
| g. Dry mass | 44.31 | 56.05 | 55.97 | 9.33 | 11.16 | 10.25 |
| of effluent | | | | | | |
| C | 15.57 | 19.09 | 19.57 | 0.95 | 1.79 | 2.10 |
| N | 0.33 | 0.29 | 0.30 | 0.43 | 0.18 | 0.64 |
| h. C/N | 46.87 | 66.78 | 64.76 | 2.19 | 9.88 | 3.26 |
| i. Total | 15.91 | 19.38 | 19.88 | 1.39 | 1.97 | 2.74 |
| (C + N) | | | | | | |
| % change | 36.96% | 39.03% | 82.77% | (26.46%) | 24.14% | (46.92%) |
| C/N | | | | | | |
| $( \frac{( h - b )}{b} 100 ) \%$ | | | | | | |
| Unaccounted | 4.77 | 2.09 | 3.47 | (0.39) | 0.75 | 1.23 |
| CNS | | | | | | |
| (c − f − i) | | | | | | |
| Unaccounted | 9.35 | 2.51 | 4.02 | (2.45) | (0.29) | 2.45 |
| total mass | | | | | | |
| (a − d − g) | | | | | | |

*Std .: Standard media with YE; AE: standard media where YE was 100% replaced with alkaline extract; SE: standard media where YE was 100% replaced with subcritical extract; Std-G: glucose omitted standard media; AE-G: YE and glucose were 100% replaced with alkaline extract; SE-G: YE and glucose were 100% replaced with subcritical extract.

Table 6 depicts on lipid fractions extracted from M. alpina biomass with help of organic solvents. Microbial cell mass cultured in CME had lower content of phospholipids (methanol fraction), i.e., 15-16% of their total lipid, whereas this was around 25% for the standard medium. Although total lipid content in cell mass was higher with SE, neutral lipid or tri-acyl glycerides (TAG in CHCl₃ fraction) was highest 68.1% with AE and these were only 47.4% and 48.3% with standard media and SE, respectively. Cell mass grown in SE was rich in glycolipids (acetone fraction) up to 27.6%, whereas these were only 7.5% in AE.

TABLE 6
Lipid class separation of M. aplina by SPE and the
percentage of individual fractions are in parenthesis.

| | Dry fungal | CHCl₃ | Acetone | MeOH |
|---|---|---|---|---|
| | biomass | Fraction | Fraction | Fraction |
| | (mg) | (mg) | (mg) | (mg) |
| Std | 108.65 ± 1.1 | 51.48 ± 0.4 | 21.73 ± 1.9 | 27.60 ± 0.58 |
| | | (47.4) | (20.0) | (25.4) |
| SE | 108.34 ± 1.6 | 52.33 ± 0.92 | 29.95 ± 2.1 | 16.81 ± 1.3 |
| | | (48.3) | (27.6) | (15.5) |
| AE | 106.06 ± 0.7 | 72.27 ± 1.7 | 7.93 ± 0.73 | 17.12 ± 1.49 |
| | | (68.1) | (7.5) | (16.1) |

Lipid class in solvent fraction: tri-acyl glycerides in CHCl₃, phospholipids in methanol (MeOH), and glycolipid in acetone.
Media type: Std.-Standard media with YE, AE-standard media where YE was 100% replaced with alkaline extract, and SE-standard media where YE was 100% replaced with subcritical extract

Table 7 presents the fatty acid profile of fungal lipid and data revealed that total of 85.4 mg, 139.6 mg, and 179.7 mg of fatty acids were obtained per g of DCM in standard media, AE, and SE medium, respectively. Highest arachidonic acid (ARA) yield of 36 mg/g of DCM was obtained in SE followed by AE and standard medium which were 24 mg/g and 21 mg/g of DCM, respectively. However, ARA percentage of total lipid fraction was higher for standard media and lowest for SE. There was substantial increase in the yield of palmitic acid, stearic acid, oleic acid, and linoleic acid when media was shifted from YE to CME, whereas a slight decrease in abundance of γ-linolenic acid was observed. Moreover, α-linolenic acid was only seen in trace amounts in the CME-supplied cell biomass and this was only 0.7 mg/g of DCM in SE (1:0) and 3.1 mg/g of DCM in AE (1:0). Although seen trace amounts, erucic, docosadienoic, and adrenic acids were also only found in CME-supplied fungal lipid extracts.

TABLE 7
Fatty acid profile of M. alpina lipid. Results are expressed in
mg/g fermented biomass and the percentage of individual
fatty acids of total fatty acids are given in the parenthesis.

| Fatty Acid (FA) | Std. | SE | AE |
|---|---|---|---|
| C14:0 (Myristic acid) | 0.7 ± 0.0 | 1.1 ± 0.1 | 3.1 ± 1.6 |
| | (0.9) | (0.6) | (2.2) |
| C15:0 | 0.7 ± 0.0 | 0.2 ± 0.0 | 0.2 ± 0.0 |
| (Pentadecanoic acid) | (0.8) | (0.1) | (0.2) |
| C16:0 (Palmitic acid) | 10.8 ± 0.5 | 17.2 ± 1.1 | 16.2 ± 0.6 |
| | (12.7) | (9.6) | (11.6) |
| C16:1 n-7 | 0.1 ± 0.0 | 0.2 ± 0.0 | 0.6 ± 0.0 |
| (Palmitoleic acid) | (0.1) | (0.1) | (0.4) |
| C17:0 | 2.3 ± 0.1 | 0.8 ± 0.1 | 0.5 ± 0.0 |
| (Heptadecanoic acid) | (2.7) | (0.5) | (0.3) |
| C16:3 n-4 | 0.6 ± 0.0 | 0.1 ± 0.1 | — |
| | (0.7) | (0.0) | |
| C18:0 (Stearic acid) | 11.6 ± 0.4 | 41.1 ± 3.2 | 14.3 ± 0.5 |
| | (13.6) | (22.9) | (10.2) |
| C18:1 n-9 (Oleic acid) | 20.0 ± 0.9 | 48.5 ± 3.3 | 38.1 ± 1.4 |
| | (23.4) | (27.0) | (27.3) |
| C18:1 n-7 | 0.1 ± 0.0 | 1.5 ± 0.1 | 6.5 ± 0.2 |
| | (0.2) | (0.8) | (4.6) |
| C18:2 n-6 C and T | 4.3 ± 0.2 | 10.4 ± 0.5 | 18.6 ± 0.7 |
| | (5.0) | (5.8) | (13.3) |
| C18:3 n-6 | 5.4 ± 0.3 | 5.3 ± 0.3 | 4.3 ± 0.2 |
| (γ-linolenic acid) | (6.3) | (3.0) | (3.1) |
| C18:3 n-4 | 0.3 ± 0.3 | — | — |
| | (0.3) | | |
| C18:3 n-3 | — | 0.7 ± 0.0 | 3.1 ± 0.1 |
| (α-linolenic acid) | | (0.4) | (2.2) |
| C20:0 (Arachidic acid) | 0.8 ± 0.0 | 2.9 ± 0.2 | 1.2 ± 0.0 |
| | (0.9) | (1.6) | (0.9) |
| C20:1 n-9 (cis-11- | 0.7 ± 0.0 | 2.3 ± 0.2 | 1.1 ± 0.0 |
| Eicosenoic acid) | (0.8) | (1.3) | (0.8) |
| C20:2 (cis-11,14- | 0.2 ± 0.0 | 0.5 ± 0.2 | 0.4 ± 0.0 |
| Eicosadienoic acid) | (0.2) | (0.3) | (0.3) |
| C20:3 n-6 (cis-8,11,14- | 3.4 ± 0.2 | 3.6 ± 0.3 | 3.3 ± 0.1 |
| Eicosatrienoic acid) | (4.0) | (2.0) | (2.3) |
| C20:4 n-6 | 21.0 ± 1.0 | 36.0 ± 2.2 | 24.0 ± 0.9 |
| (Arachidonic acid) | (24.6) | (20.0) | (17.2) |
| C22:0 (Behenic acid) | 1.2 ± 0.0 | 4.1 ± 0.4 | 1.9 ± 0.1 |
| | (1.4) | (2.3) | (1.4) |
| C22:1 n-9 (Erucic acid) | — | 0.1 ± 0.1 | — |
| | | (0.1) | |
| C22:2 n-3 | — | 0.3 ± 0.0 | 0.1 ± 0.1 |
| (Docosadienoic acid) | | (0.2) | (0.0) |
| C22:4 n-6 | — | 0.2 ± 0.0 | — |
| | | (0.1) | |
| C24:0 (Lignoceric acid) | 1.3 ± 0.0 | 2.7 ± 0.3 | 1.9 ± 0.1 |
| | (1.5) | (1.5) | (1.4) |
| Total FA in Sample mg/g | 85.4 ± 3.6 | 179.7 ± 12.2 | 139.6 ± 3.7 |
| (% of total FA) | (100.0) | (100.0) | (100.0) |

Std.- Standard media with YE, AE-standard media where YE was 100% replaced with alkaline extract, and SE-standard media where YE was 100 % replaced with subcritical extract

Effect of substrate type on the production of ARA by M. alpina has been discussed previously (Aki et al., 2001; Ghobadi et al., 2022; Higashiyama et al., 2002; Totani et al., 2000). Oil seed or meal have been considered as potential candidates to increase ARA productivity of the fungus. Ghobadi et al. (2022) compared several oil cakes from sunflower, soybean, colza, and olives, upon which sunflower cake provided high yield of ARA followed by soybean and these results were related with higher proportion of unsaturated fatty acids in the substrate. Aki et al. (2001) considered both YE and soybean meal as suitable supplements for ARA production. Park et al. (1999) compared YE, soybean meal, gluten meal, and corn steep liquor as a medium supplement for M. alpina. It was reported that soybean meal provided better morphological structure to fungus and thus the authors concluded that morphology had a direct relation on ARA productivity (Park et al., 1999).

Higher ARA per g of DCM with its relatively low fraction of total fatty acids with CME could be potentially linked with ease of fungal access to nitrogen in media, as proteins here would be more structurally more-simple as compared to YE. Previous literature has discussed the beta-oxidation of long chain PUFA during nitrogen limitation and rechanneling the carbon skeleton for synthesis of C16-C18 fatty acid synthesis (Lu et al., 2020), something that could possibly explain higher yield of palmitic acid, stearic acid, oleic acid, and linoleic acid in our study when media as shifted from YE to CME. Further, availability of metabolic nitrogen could even explain the low phospholipid fraction in total lipid for fungal biomass supplied with CME. It was found that nitrogen limitation leads to increased phospholipase activity, thus degrading the phospholipids and suppling acyl chains for TAG biosynthesis (Lu et al., 2020).

Beside ALA oleic acid was one of the major fatty acids detected in the lipid extracted from all three treatment groups followed by stearic acid and palmitic acid. Even though oleic acid (C18:1 n-9 cis) and elaidic acid (C18:1 n-9 trans) have identical retention time in current GC, the peak detected was identified as oleic acid based on the biosynthetic pathway of fatty acids described in M. alpina by Sakuradani, (2010). It is worth to mention here that polyunsaturated fatty acids (PUFAs) and monounsaturated fatty acids (MUFAs) including oleic acid reported to have a cardio-protective role (Balta et al., 2021). Oleic acid also possessed antitumor effects (Carrillo et al., 2012), it showed proliferation inhibition in different tumor cell lines including prostate carcinoma PC-3 cells (Hughes-Fulford et al., 2001). Oleic acid concentration in the lipid fraction were high as 23% and 27% in biomass in YE and CME culture, respectively. Further this fraction was highest in biomass culture in CME as compared any other fatty acid.

Table 8 shows the amino acid profile in a canola meal extract obtained by the methods described herein:

TABLE 8
Amino Acid Profile in CME-18 primary

| | % in | Std. |
|---|---|---|
| Amino Acids | CME | dev. |
| Aspartic Acid | 3.54% | 0.04% |
| Glutamic Acid | 11.64% | 0.05% |
| Serine | 2.43% | 0.01% |
| Glycine | 2.74% | 0.01% |
| Histidine | 1.53% | 0.08% |
| Arginine | 2.51% | 0.10% |
| Threonine | 1.92% | 0.08% |
| Alanine | 2.69% | 0.11% |
| Proline | 4.61% | 0.20% |
| Tyrosine | 1.44% | 0.10% |
| Valine | 2.34% | 0.00% |
| Methionine | 2.39% | 0.51% |
| Cystine | 1.61% | 0.17% |
| Isoleucine | 1.78% | 0.01% |
| Leucine | 3.34% | 0.02% |
| Phenylalanine | 2.32% | 0.10% |
| Lysine | 2.97% | 0.13% |
| Tryptophan | 0.73% | 0.07% |
| Total Amino | 52.54% | 1.78% |
| Acids | | |

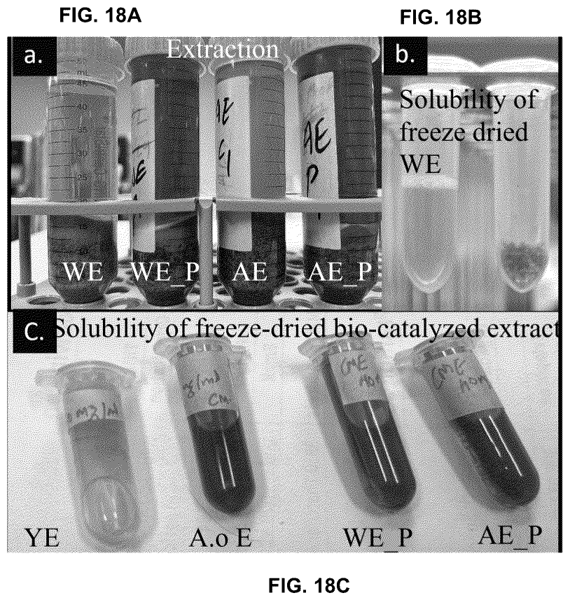

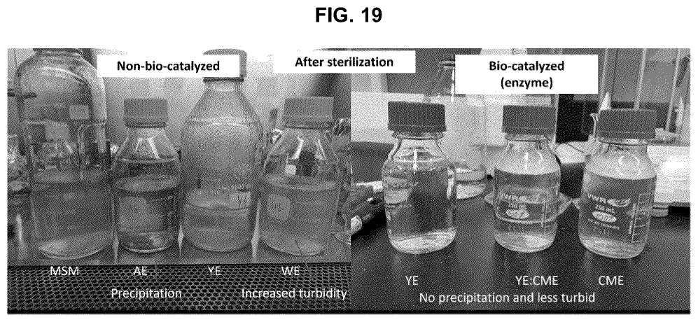

FIGS. 18 and 19 compare the results of biocatalyzed vs. non-biocatalysed extractions. FIG. 18A shows extraction of canola meal with and without enzymatic catalysis, FIG. 18B shows the solubility of 40 mg of freeze dried WE in 1 ml of water, where the turbidity or haze formation in the solution can be seen and FIG. 18C shows that bio-catalyzed extracts are less hazy when mixed with same amount in water.

Further, FIG. 19 shows enzyme catalyzed canola meal has can have better properties as compared to non-catalyzed water or alkaline extracted canola meal. This can be seen in the precipitation and increased turbidity in AE and WE, respectively, after sterilization in 121° C. for 15 min, whereas enzyme catalyzed extract remains clear without precipitation. Without being bound by theory, this may be due to presence of hydrolyzed proteins in enzyme digested meal which can be further clarified by SDS PAGE as shown in FIG. 8.

### SUMMARY

In summary, enzymatic extraction led to high yield of extract with increased concentration of protein hydrolysate of around 70% as compared to other methods. Enzymes also increased the solubility of the extracts and significantly reduced protein precipitation on medium sterilization. Moreover, CME in culture contributed to the growth of microbial cultures and stimulated their productivity. Up to 50% replacement of YE with chemically extracted CME showed optimal result for bacteria, whereas enzyme hydrolyzed could replace this to 100%. On the other hand, fungus could efficiently utilize chemically catalyzed CME. While comparing alkaline and subcritical extracts of canola meal, the alkaline extract had positive effect on growth, whereas sub-critical extract significantly improved lipid and ARA content of the microbial biomass. While CME supported fungal growth, we observed growth was optimal in YE. To sum up, lab-based results show high potential for CME to be commercialized as microbial medium supplement.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present description is found to be defined differently in a document incorporated herein by reference, the definition provided herein is to serve as the definition for the term.

### CITATIONS FOR REFERENCES REFERRED TO IN THE SPECIFICATION

Aki, T., Nagahata, Y., Ishihara, K., Tanaka, Y., Morinaga, T., Higashiyama, K., Akimoto, K., Fujikawa, S., Kawamoto, S., Shigeta, S., Ono, K., & Suzuki, O. (2001). Production of arachidonic acid by filamentous fungus, Mortierella alliacea strain YN-15. Journal of the American Oil Chemists' Society, 78 (6), 599-604.
Arora, M., 2022. Cell Culture Media: A Review. Materials and Methods.
Balta, I., Stef, L., Pet, I., Iancu, T., Stef, D., & Corcionivoschi, N. (2021). Essential Fatty Acids as Biomedicines in Cardiac Health. Biomedicines, 9 (10), Article 10.
BioBasic, n.d. Yeast Extract—Bio Basic [WWW Document]. URL https://www.biobasic.com/yeast-extract (accessed 3.3.22).
Boling-Frankenbach, S. D., Peter, C. M., Douglas, M. W., Snow, J. L., Parsons, C. M., Baker, D., 2001. Efficacy of Phytase for Increasing Protein Efficiency Ratio Values of Feed Ingredients. Poultry Science 80, 1578-1584.
Bradford, M. M. (1976). A rapid and sensitive method for the quantitation of microgram quantities of protein utilizing the principle of protein-dye binding. Analytical Biochemistry, 72(1), 248-254.
Canola Council, 2022. Canadian canola export statistics [WWW Document]. The Canola Council of Canada. URL https://www.canolacouncil.org/markets-stats/exports/(accessed 3.22.22).
Carrillo, C., Cavia, M. D. M., & Alonso-Torre, S. R. (2012). Antitumor effect of oleic acid; mechanisms of action: A review. Nutricion Hospitalaria, 27 (6), 1860-1865.
Deleu, L. J., Lambrecht, M. A., Van de Vondel, J., Delcour, J. A., 2019. The impact of alkaline conditions on storage proteins of cereals and pseudo-cereals. Current Opinion in Food Science, Food Chemistry and Biochemistry⋅Food Bioprocessing 25, 98-103.
Doran, P. M. (2013). Chapter 12—Homogeneous Reactions. In P. M. Doran (Ed.), Bioprocess Engineering Principles (Second Edition) (pp. 599-703). Academic Press.
Emerson, D., Tang, J., 2007. Nutrition and Media, in: Methods for General and Molecular Microbiology. John Wiley & Sons, Ltd, pp. 200-214.
Engles, C., Schieber, A., Ganzle, M. G., 2012. Sinapic acid derivatives in defatted Oriental mustard (Brassica juncea L.) seed meal extracts using UHPLC-DAD-ESI-MS and identification of compounds with antibacterial activity. European Food Research and Technology 234:535-542.
Ghobadi, Z., Hamidi-Esfahani, Z., & Azizi, M. H. (2022). Statistical optimization of arachidonic acid synthesis by Mortierella alpina CBS 754.68 in a solid-state fermenter. Food Science & Nutrition, 10 (2), 436-444.
Godard, P., Urrestarazu, A., Vissers, S., Kontos, K., Bontempi, G., van Helden, J., & André, B. (2007). Effect of 21 different nitrogen sources on global gene expression in the yeast Saccharomyces cerevisiae. Molecular and Cellular Biology, 27 (8), 3065-3086.
Higashiyama, K., Fujikawa, S., Park, E. Y., & Shimizu, S. (2002). Production of arachidonic acid by Mortierella fungi. Biotechnology and Bioprocess Engineering, 7 (5), 252-262.
Higashiyama, K., Yaguchi, T., Akimoto, K., Fujikawa, S., & Shimizu, S. (1998). Enhancement of arachidonic acid production by Mortierella alpina 1S-4. Journal of the American Oil Chemists' Society, 75 (11), 1501-1505.
Hughes-Fulford, M., Chen, Y., & Tjandrawinata, R. R. (2001). Fatty acid regulates gene expression and growth of human prostate cancer PC-3 cells. Carcinogenesis, 22 (5), 701-707.
Kuenz, A., 2021. Substitution of yeast extract in industrially relevant bioprocesses (SubBioPro).
Lähn, K., Wolf, G., Ullrich-Eberius, C., & Koch, E. (2002). Cultural characteristics and in vitro antagonistic activity of two isolates of Mortierella alpina Peyronel/Kulturmerkmale und in vitro antagonistische Aktivität zweier Isolate von Mortierella alpina Peyronel. Zeitschrift Für Pflanzenkrankheiten Und Pflanzenschutz/Journal of Plant Diseases and Protection, 109 (2), 166-179.
Law, J. H., & Slepecky, R. A. (1961). Assay of poly-beta-hydroxybutyric acid. Journal of Bacteriology, 82 (1), 33-36.
Leite, J. A. S., Montoya, C. A., Loveday, S. M., Maes, E., Mullaney, J. A., McNabb, W. C., & Roy, N. C. (2021). Heat-Treatments Affect Protease Activities and Peptide Profiles of Ruminants' Milk. Frontiers in Nutrition, 8, 626475.
Liu, Q., Wu, L., Pu, H., Li, C., & Hu, Q. (2012). Profile and distribution of soluble and insoluble phenolics in Chinese rapeseed (Brassica napus). Food Chemistry, 135 (2), 616-622.
Lu, H., Chen, H., Tang, ×., Yang, Q., Zhang, H., Chen, Y. Q., & Chen, W. (2020). Time-resolved multi-omics analysis reveals the role of nutrient stress-induced resource reallocation for TAG accumulation in oleaginous fungus Mortierella alpina. Biotechnology for Biofuels, 13 (1), 116.
Lv, G., Xu, Y., Tu, Y., Cheng, X., Zeng, B., Huang, J., & He, B. (2021). Effects of Nitrogen and Phosphorus Limitation on Fatty Acid Contents in Aspergillus oryzae. Frontiers in Microbiology, 12.
Maenz, D. D, 2007. The production and use of High-valued canola protein concentrates. Feed and Industrial Raw Material-Feed. In proceedings of the 12^{th} International Rapeseed Congress, Volume 7, Mar. 26-30, 2007, Wuhan, China, 274-276. Gcirc.org/fileadmin/documents/Proceedings/IRCWuhan2007% 20vol5/5-146-149.pdf#page=290 Qiang, Y., 2019. Why fermentation industry prefers baker's yeast extract? Microbial Nutrition [WWW Document]. URL https://en.angelyeast.com/blog/microbial-nutrition/why-fermentation-industry-prefer-baker-yeast-extract.html (accessed 10.4.2022).
Mandal, M., Misra, D., Ghosh, N. N., & Mandal, V. (2017). Physicochemical and elemental studies of Hydrocotyle javanica Thunb. For standardization as herbal drug. Asian Pacific Journal of Tropical Biomedicine, 7 (11), 979-986.
Marudkla, J., Lee, W.-C., Wannawilai, S., Chisti, Y., & Sirisansaneeyakul, S. (2018). Model of acetic acid-affected growth and poly (3-hydroxybutyrate) production by Cupriavidus necator DSM 545. Journal of Biotechnology, 268, 12-20.
Miller, G. L. (1959). Use of Dinitrosalicylic Acid Reagent for Determination of Reducing Sugar. Analytical Chemistry, 31 (3), 426-428.
Ozimek, E., & Hanaka, A. (2021). Mortierella Species as the Plant Growth-Promoting Fungi Present in the Agricultural Soils. Agriculture, 11 (1), Article 1.
Park, E. Y., Koike, Y., Higashiyama, K., Fujikawa, S., & Okabe, M. (1999). Effect of nitrogen source on mycelial morphology and arachidonic acid production in cultures of Mortierella alpina. Journal of Bioscience and Bioengineering, 88 (1), 61-67.
Patel, A. S., Kar, A., Pradhan, R. C., Mohapatra, D., & Nayak, B. (2019). Effect of baking temperatures on the proximate composition, amino acids and protein quality of deoiled bottle gourd (Lagenaria siceraria) seed cake fortified biscuit. LWT, 106, 247-253.
Perera, S. P., McIntosh, T. C., & Wanasundara, J. P. D. (2016). Structural Properties of Cruciferin and Napin of Brassica napus (Canola) Show Distinct Responses to Changes in pH and Temperature. Plants, 5 (3), 36.
Preece, K. E., Hooshyar, N., Krijgsman, A. J., Fryer, P. J., & Zuidam, N. J. (2017). Intensification of protein extraction from soybean processing materials using hydrodynamic cavitation. Innovative Food Science & Emerging Technologies, 41, 47-55.
SAKURADANI, E. (2010). Advances in the Production of Various Polyunsaturated Fatty Acids through Oleaginous Fungus Mortierella alpina Breeding. Bioscience, Biotechnology, and Biochemistry, 74 (5), 908-917.
Sereewatthanawut, I., Prapintip, S., Watchiraruji, K., Goto, M., Sasaki, M., & Shotipruk, A. (2008). Extraction of protein and amino acids from deoiled rice bran by subcritical water hydrolysis. Bioresource Technology, 99 (3), 555-561.
Sigma-Aldrich, n.d. Yeast Extract-Select Yeast Extract, Yeast extract [WWW Document]. URL https://www.sigmaaldrich.com/CA/en/substance/yeastextract123458013012 (accessed 3.3.22).
Smith, A. M., & Agiza, A. H. (1951). The determination of amino-acids colorimetrically by the ninhydrin reaction. Analyst, 76 (908), 623-627.
Sperotto, G., Stasiak, L. G., Godoi, J. P. M. G., Gabiatti, N. C., De Souza, S. S., 2021. A review of culture media for bacterial cellulose production: complex, chemically defined and minimal media modulations. Cellulose 28, 2649-2673.
Tao, Z., Yuan, H., Liu, M., Liu, Q., Zhang, S., Liu, H., Jiang, Y., Huang, D., & Wang, T. (2023). Yeast Extract: Characteristics, Production, Applications and Future Perspectives. Journal of Microbiology and Biotechnology, 33 (2), 151-166.
Totani, N., Hyodo, K., Yamaguchi, A., & Ueda, T. (2000). A Study on New Nitrogen Source for Cultivation of Genus Mortierella. Journal of Japan Oil Chemists' Society, 49 (5), 479-485,514.
Vieira, E. F., Carvalho, J., Pinto, E., Cunha, S., Almeida, A. A., Ferreira, I. M. P. L. V. O., 2016. Nutritive value, antioxidant activity and phenolic compounds profile of brewer's spent yeast extract. Journal of Food Composition and Analysis 52, 44-51.
Wang, M., Yang, C., François, J. M., Wan, X., Deng, Q., Feng, D., Deng, S., Chen, S., Huang, F., Chen, W., & Gong, Y. (2021). A Two-step Strategy for High-Value-Added Utilization of Rapeseed Meal by Concurrent Improvement of Phenolic Extraction and Protein Conversion for Microbial Iturin A Production. Frontiers in Bioengineering and Biotechnology, 9.
Weder, J. K., & Sohns, S. (1983). Model studies on the heating of food proteins. Amino acid composition of lysozyme, ribonuclease and insulin after dry heating. Zeitschrift Fur Lebensmittel-Untersuchung Und-Forschung, 176 (6), 421-425.
Wickramasuriya, S. S., Yi, Y.-J., Yoo, J., Kang, N. K., Heo, J. M., 2015. A review of canola meal as an alternative feed ingredient for ducks. Journal of Animal Science and Technology 57, 29. Yaakob, M. A., Mohamed, R. M. S. R., Al-Gheethi, A., Aswathnarayana Gokare, R., & Ambati, R. R. (2021). Influence of Nitrogen and Phosphorus on Microalgal Growth, Biomass, Lipid, and Fatty Acid Production: An Overview. Cells, 10 (2), 393.

1. A canola meal extract (CME) comprising 20-80% protein, 10-30% carbohydrates, 5-30% fermentable sugars, 1-10% lipids, 1-10% minerals, and 50-300 μg phenolics per g dry weight.

2. The CME according to claim 1, further comprising 50-200 mg of vitamins per kg dry weight, optionally wherein the vitamins comprise one or more selected from the group consisting of vitamin K, niacin, riboflavin, thiamine, biotin, pyridoxine, panthothenate and choline.

3. The CME according to claim 1, wherein the minerals comprise one or more selected from the group consisting of P, K, S, Se, Cu, Fe, Mn, Ca, Mg, and Zn.

4. The CME according to claim 1, further comprising less than 2% other impurities.

5. A culture medium comprising the CME according to claim 1.

6. The culture medium according to claim 5, further comprising one or more additional extracts, optionally wherein the one or more additional extracts comprise yeast extract, beef extract, peptone, tryptone, or soytone, or a combination thereof.

7. A method of promoting microbial growth or survival, the method comprising contacting the CME according to claim 1 in a culture with at least one microbe.

8. The method of claim 7, wherein the at least one microbe comprises a bacterium, an archaeum, a fungus, an alga, or a protozoan, or a combination thereof.

9. The method of claim 8, wherein
the bacterium comprises a carboxydotroph, optionally of genus Cupriavidus, optionally Cupriavidus necator,
the bacterium comprises an acetogen, optionally of genus Clostridium, optionally Clostridium carboxidivorans;
the bacterium produces lactic acid, optionally wherein the bacterium comprises the genus Lactobacillus, Leuconostoc, Pediococcus, Lactococcus, or Streptococcus or combinations thereof, optionally Lactobacillus rhamnosus or Lactobacillus acidophilus;
the bacterium comprises a coliform, optionally of genus Escherichia, optionally Escherichia coli;
the fungus comprises an oleaginous fungus, optionally of genus Mortierella, optionally Mortierella alpina fungus comprises genus Saccharomyces, optionally Saccharomyces cerevisiae; or
the fungus comprises a saccharifying mold, optionally of genus Aspergillus, optionally Aspergillus oryzae, or Aspergillus niger, or a combination thereof.

10. A method of promoting growth or survival of at least one mammalian cell, the method comprising contacting the CME according to claim 1 in a culture with the at least one mammalian cell, for promoting growth or survival of the at least one mammalian cell.

11. A method of producing a canola meal extract (CME) comprising:
combining canola meal with a fluid to form a mixture;
optionally raising the pH of the mixture to between 9 and 12;
heating the mixture to a temperature between 20° C. and 170° C.;
catalyzing the mixture; and
obtaining the CME from the mixture.

12. The method of claim 11, wherein raising the pH of the mixture comprises adding a base, optionally NaOH, to the mixture.

13. The method of claim 11, wherein the catalyzing comprises treating the mixture with at least one enzyme, wherein the at least one enzyme comprises a proteolytic enzyme, optionally wherein the proteolytic enzyme comprises papain, pepsin, alkaline protease, trypsin, Alcalase or a combination thereof.

14. The method of claim 13, wherein the catalyzing comprises treating the mixture with the at least one enzyme for between 2 and 96 hours, optionally with continuous agitation, optionally at a temperature between 20° C. and 90° C. and optionally between pH 3 and 12.

15. The method of claim 11, wherein the catalyzing comprises treating the CME with at least one fungus, optionally wherein the step of catalyzing is performed prior to the steps of optionally raising the pH of the mixture and heating the mixture.

16. The method of claim 11, where the catalyzing comprises inherent lactic fermentation, optionally wherein the step of catalyzing is performed prior to the steps of optionally raising the pH of the mixture and heating the mixture.

17. The method of claim 11, wherein the steps of optionally raising the pH of the mixture to between 9 and 12 and heating the mixture to a temperature between 20° C. and 170° C. are performed under continuous agitation.

18. The method of claim 11, further comprising at least one pretreatment step, optionally defatting or sterilization of the canola meal.

19. The CME produced by the method of claim 11.

20. A kit comprising the CME of claim 1 and a culture medium.
